(19) United States
(12) Patent Application Publication
(10) Pub. No.: US 2026/0341467 A1
(43) Pub. Date: Oct. 29, 2026

(54) DIETARY COMPOSITIONS FOR PROMOTING AUTOPHAGY IN AN INTERMITTENT FASTING-MIMICKING DIET, METHODS AND KITS

(71) Applicants: Valter D. LONGO, Playa del Rey, CA (US); L-NUTRA INC., Plano, TX (US)

(72) Inventors: William HSU, Chestnut Hill, MA (US); Valter D. LONGO, Playa del Rey, CA (US)

(73) Assignees: Valter D. LONGO, Playa del Rey, CA (US); L-NUTRA INC., Plano, TX (US)

(21) Appl. No.: 19/659,096

(22) Filed: Apr. 27, 2026

Related U.S. Application Data
(60) Provisional application No. 63/795,693, filed on Apr. 28, 2025.

(51) Int. Cl. G16H 20/60; A23L 33/00

(52) U.S. Cl. CPC G16H 20/60; A23L 33/30

(57) ABSTRACT

An autophagy-inducing, fasting-mimicking diet (FMD) is provided in the form of multi-day dietary regimens, compositions, foodstuffs, and kits operable to induce autophagy and improve metabolic health in mammalian subjects while allowing controlled nutrient intake. The regimens include a first-day dietary formulation providing about 300 to 1600 kcal with proteins, fats, and carbohydrates distributed within defined macronutrient ranges, followed by at least one subsequent-day dietary formulation providing about 200 to 1000 kcal with macronutrient ranges configured to sustain fasting-like metabolism and autophagic flux. In certain embodiments, a five-day regimen provides about 1100 kcal on Day 1 with approximately 11% protein kcal, 46% fat kcal, and 43% carbohydrate kcal, and about 720 kcal on Days 2-5 with approximately 9% protein kcal, 44% fat kcal, and 47% carbohydrate kcal, or an alternative formulation with about 9% protein kcal, 61% fat kcal, and 30% carbohydrate kcal on Day 1 and about 8% protein kcal, 52% fat kcal, and 40% carbohydrate kcal on Days 2-5. Foodstuffs manufactured from the disclosed compositions can include nutrition bars, soups, snacks, beverages, teas, and supplements, assembled into pre-portioned daily kits labeled for sequential consumption over the regimen. In a randomized clinical study in healthy adults, regimens within the disclosed macronutrient ranges significantly increased ketone bodies and improved metabolic markers including fasting glucose, insulin, IGF-1, and HOMA-IR compared to control, and at least one formulation increased autophagic flux in peripheral blood mononuclear cells as measured by the chloroquine-treated to untreated LC3B-II/LC3B-I ratio, supporting use of the FMD regimens as non-pharmacologic interventions to modulate autophagy, promote metabolic health, and reduce risk of age-related diseases.

### CROSS-REFERENCE TO RELATED APPLICATIONS

This application claims the benefit of U.S. Provisional Application No. 63/795,693 filed Apr. 28, 2025, the entire contents of which are incorporated herein by reference.

### BACKGROUND

### Technical Field

The present disclosure relates to dietary compositions, foodstuffs, methods, and kits for inducing autophagy in mammalian subjects as part of a fasting-mimicking diet (FMD), and to methods of using such compositions, foodstuffs, and kits to modulate autophagic flux and metabolic health markers in human subjects. The disclosed dietary compositions, foodstuffs, and methods are operable to induce autophagy in mammalian subjects thereby modulating metabolic pathways and cellular homeostasis, including but not limited to autophagic flux, insulin sensitivity, and ketone metabolism. The disclosed dietary compositions, foodstuffs and methods are useful for promoting longevity, improving metabolic health, and preventing age-related diseases.

### Background

Autophagy is a lysosomal-mediated degradation cellular process that degrades and recycles intracellular components, such as damaged organelles, misfolded proteins, and other intracellular components to maintain cellular homeostasis. Dysregulation of autophagy has been implicated in numerous pathological conditions, including, for example, metabolic disorders, neurodegenerative diseases, cardiovascular diseases, cancer and age-related decline. Autophagic flux, reflected by the rate at which cells degrade and recycle their own components through the autophagy pathway, provides insight into the dynamic process of autophagosome formation, maturation, and degradation, and is measured, for example, as the ratio of LC3B-II to LC3B-I protein in chloroquine-treated cells versus untreated cells. Measuring autophagic flux can further provide insight into various diseases by providing insight into how a cell is cleaning-up damaged materials. Because autophagy supports cellular maintenance and resilience, interventions that promote autophagic flux have the potential to improve metabolic health for subjects, enhance longevity, and mitigate the progression of age-related diseases.

Dietary restriction, including intermittent fasting (IF) and fasting-mimicking diets (FMD), has emerged as a promising non-pharmacologic strategy to modulate nutrient-sensing pathways such as mTOR and AMPK, to promote autophagy and improve metabolic health. However, conventional fasting regimens are often difficult to adhere to, and prior to the present disclosure there was a lack of evidence that autophagy could be robustly induced in human subjects while still undergoing a calorie-based diet with defined macronutrient profiles rather than complete food deprivation.

A fasting-mimicking and enhancing diet includes a diet that is operable to mimic the effects of fasting (e.g., fast mimicking) typically by providing a subject with at most 50% of their normal caloric intake. However, no credible studies exist that demonstrate autophagy can be achieved while still undergoing a calorie-based diet, regardless of macronutrient profiles.

Accordingly, there is a need for dietary compositions, foodstuffs, structured meal regimens, and kits that (i) mimic the metabolic effects of fasting while providing controlled calories and essential nutrients, (ii) are formulated within defined macronutrient ranges that reproducibly induce metabolic conditions associated with autophagy, and (iii) are supported by human clinical data demonstrating effects on autophagic flux and metabolic health markers.

### SUMMARY

Disclosed are autophagy-inducing dietary compositions, foodstuffs, methods and kits that promote inducing metabolic conditions associated with autophagy in a subject (e.g., mammalian subjects including, but not limited to, primates (including higher primates), sheep, dog, rodents, guinea pig, goat, pig, cat, rabbit and cow) and are fasting-mimicking. The autophagy-inducing dietary compositions may induce metabolic conditions associated with autophagy in a human subject while achieving a fasting-mimicking state for a subject while providing controlled nutrient intake over a multi-day regimen. The autophagy-inducing dietary compositions may also include a first-day dietary formulation containing 300 to 1600 kcal, with a protein component, fat component, and carbohydrate component distributed in ranges that can initiate metabolic shifts associated with fasting and autophagy. The first-day dietary formulation is an autophagy-inducing dietary composition, or group of foodstuffs, intended for consumption by a subject on a first day of a multi-day FMD regimen, within the caloric and macronutrient ranges described herein. The first-day dietary formulation may be followed by a subsequent-day dietary formulation for at least one additional day containing from 200 to 1000 kcal, with macronutrient distributions that may sustain fasting-induced metabolic effects, including inhibition of mTOR, activation of AMPK, and stimulation of ketogenesis. The subsequent-day dietary formulation is an autophagy-inducing dietary composition, or group of foodstuffs, intended for consumption on one or more days following the first day of the multi-day FMD regimen, within the caloric and macronutrient ranges described herein. Each autophagy-inducing dietary composition may be operable, alone or in combination with other autophagy-inducing dietary compositions in a group, to promote metabolic conditions associated with autophagy when consumed by a subject as part of a structured dietary regimen.

A multi-day dietary regimen of consuming autophagy-inducing dietary compositions as part of a FMD promoting autophagy is also contemplated that may span at least two days, and in other examples a multi-day dietary regimen may span four or five days. The different multi-day dietary regimens of consuming the disclosed autophagy-inducing dietary compositions, and foodstuffs prepared based on the autophagy-inducing dietary compositions, provide the subject flexibility in achieving fasting-related metabolic effects from consuming products based on the disclosed autophagy-inducing dietary compositions. The macronutrient composition for each day of the multi-day dietary regimen may be controlled, with the protein component comprising from 1% to 20% of the total composition kcal on the first day and from 1% to 15% of the total composition kcal on subsequent day(s), fat component comprising from 30% to 80% of the total composition kcal on the first day and from 30% to 75% of the total composition kcal on subsequent day(s), and carbohydrate component comprising from 10% to 60% of the total composition kcal on the first day and from 15% to 55% of the total composition kcal on subsequent day(s). One or more macronutrients may be provided, with the protein, fat, and carbohydrate content falling within optimized kcal percentages of the total kcal of the autophagy-inducing dietary composition, allowing for adaptation based on individual needs of a subject or subject preferences.

Disclosed are also methods of inducing autophagy in a subject. The autophagy inducing methods may include administering foodstuffs based on the disclosed autophagy-inducing dietary compositions over a multi-day dietary regimen. For example, a subject may consume a first-day dietary formulation followed by at least one subsequent-day dietary formulation that is different than the first day formulation, leading to metabolic shifts of the subject that may include ketogenesis, suppression of insulin and IGF-1, and activation of nutrient-sensing pathways linked to autophagy. In certain examples, the dietary regimen may be repeated periodically, such as monthly or bimonthly, to sustain long-term metabolic benefits for the subject.

Methods of manufacturing one or more autophagy-inducing dietary compositions are also disclosed. The methods may include selecting and combining ingredients for the autophagy-inducing dietary compositions to achieve a macronutrient formulation that aligns with the target autophagy-inducing dietary composition, followed by processing the ingredients into foodstuffs. Suitable foodstuffs include, but are not limited to, nutrition bars, soups, powders, beverages, snacks, teas, and/or supplements. The manufacturing process may incorporate low-temperature dehydration, freeze-drying, cold extrusion, vacuum drying, or spray-drying to help maintain bioactive compound integrity and optimize food stability. The foodstuffs can be pre-portioned and organized into individual daily packages, with a daily package optionally labeled for sequential consumption over the multi-day dietary regimen. The use of pre-portioned servings of one or more foodstuffs, may increase subject adherence to the dietary protocol.

A dietary kit of foodstuffs formed from the autophagy-inducing dietary compositions is also disclosed. The dietary kit may include one or more pre-portioned autophagy-inducing dietary compositions. The dietary kit is operable to promote fasting-mimicking and autophagy. The foodstuffs made from the autophagy-inducing dietary compositions in the dietary kit can be organized into individual daily packages, with each daily package optionally labeled for sequential consumption over the multi-day dietary regimen. The dietary kit may be operable to maintain dietary compliance of a subject while supporting the stability of macronutrient distribution of foodstuffs. Packaging may include vacuum-sealing, nitrogen flushing, or multi-layer oxygen-barrier films to reduce oxidation and moisture intrusion, thereby helping to preserve nutrient integrity and extend shelf life.

Clinical studies described herein demonstrate that multi-day FMD regimens using disclosed macronutrient ranges increase autophagic flux in human peripheral blood mononuclear cells for at least one formulation and significantly improve multiple metabolic health markers in FMD groups compared to control diet.

A structured dietary intervention is disclosed that may enable individuals to experience the physiological benefits of fasting—particularly an autophagy induction and metabolic optimization—without requiring complete food deprivation. The compositions, kits, method of use, and manufacturing process may collectively establish a flexible, repeatable dietary regimen that supports cellular repair, metabolic health, and longevity in a practical and consumer-friendly format.

Both the foregoing general description and the following detailed description are exemplary and explanatory only and are not restrictive of the disclosed examples, as claimed.

### INCORPORATION BY REFERENCE

All publications, patents, and patent applications mentioned in this specification are herein incorporated by reference to the same extent as if each individual publication, patent, or patent application was specifically and individually indicated to be incorporated by reference, except for any definitions, disclaimers, disavowals, and inconsistencies, to the maximum extent allowable by law.

US 2011/0118528 A1 by Longo for Methods and Nutritional Formulations to Increase the Efficacy and Reduce the Side Effects of Cancer Treatment published May 19, 2011;

US 2013/0045215 A1 by Longo for Treatments Related to GH/IGF-1 Axis Inhibition published Feb. 21, 2013;

US 2013/0316948 A1 by Longo for Growth Hormone Receptor Deficiency Causes a Major Reduction in Pro-Aging Signaling, Cancer and Diabetes in Humans, published Nov. 28, 2013;

U.S. Pat. No. 8,211,700 by Longo for Induction of Differential Stress Resistance and Uses thereof, issued Jul. 3, 2012;

U.S. Pat. No. 8,856,646 by Longo for dietary compositions and Methods for Protection Against Chemotherapy, Radiotherapy, Oxidative Stress, and Aging issued Oct. 21, 2014;

U.S. Pat. No. 9,237,761 by Longo for Methods and Diets for Lowering Glucose and/or IGF-1 Levels issued Jan. 19, 2016;

U.S. Pat. No. 9,386,790 by Longo for Fasting Condition as Dietary Treatment of Diabetes, issued Jul. 12, 2016; and

U.S. Pat. No. 10,015,980 by Longo for Methods and Formulations Promoting Tissue/Organ Regeneration, Longevity and Healthspan issued Jul. 10, 2018; and

U.S. Pat. No. 10,172,839 by Longo for Use of Short Term Starvation Regimen in Combination with Kinase Inhibitors to Enhance Traditional Chemo-Drug Efficacy and Feasibility and Reverse Side Effects of Kinases in Normal Cells and Tissues issued Jan. 8, 2019.

### BRIEF DESCRIPTION OF THE DRAWINGS

The novel features of the teachings are set forth with particularity in the present disclosure. A better understanding of the features and advantages will be obtained by reference to the following detailed description that sets forth illustrative examples, in which the principles of the disclosed teachings are utilized, and the accompanying drawings of which:

FIG. 1 is a table summarizing macronutrient profiles for the autophagy-inducing dietary compositions on a first and subsequent days;

FIG. 2A is a table illustrating protein components for the autophagy-inducing dietary compositions;

FIG. 2B is a table illustrating fat components for the autophagy-inducing dietary compositions;

FIG. 2C is a table illustrating carbohydrate components for the autophagy-inducing dietary compositions;

FIG. 3A is a table illustrating macronutrient percentages for autophagy-inducing dietary compositions designed to be consumed as part of a structured multi-day dietary regimen of 1100 kcal on Day 1;

FIG. 3B is a table illustrating macronutrient percentages for autophagy-inducing dietary compositions designed to be consumed as part of a structured multi-day dietary regimen that provides approximately 720 kcal per day from Days 2 to 5;

FIG. 4A is a table illustrating alternative macronutrient percentages for autophagy-inducing dietary compositions designed to be consumed as part of a structured multi-day dietary regimen of 1100 kcal on Day 1;

FIG. 4B is a table illustrating alternative macronutrient percentages for autophagy-inducing dietary compositions designed to be consumed as part of a structured multi-day dietary regimen that provides approximately 720 kcal per day from Days 2 to 5; and

FIGS. 5A-5H are graphs showing clinical test data from a randomized trial in human subjects, including trends in metabolic health markers and autophagic flux across intervention groups and study visits.

### DETAILED DESCRIPTION

It has been found that fasting is an effective natural trigger of autophagy, as fasting activates key nutrient-sensing pathways that regulate cellular energy balance. Disclosed are dietary kits comprises of one or more foodstuffs manufactured from autophagy-inducing dietary compositions. Also disclosed are methods of manufacturing autophagy-inducing compositions, foodstuffs, and kits. The disclosed foodstuffs, autophagy-inducing dietary compositions and methods can be part of an FMD. The multi-day dietary regimen comprises a low-calorie, low-protein, high-unsaturated fat regimen.

The autophagy-inducing dietary compositions and foodstuffs are formulated to mimic the metabolic effects of fasting while providing essential nutrients that support cellular homeostasis for the subject. Moreover, the autophagy-inducing composition formulations are effective at triggering autophagic flux through nutrient-sensing pathways, including inhibition of the mechanistic target of rapamycin (“mTOR”), activation of AMP-activated protein kinase (“AMPK”), and upregulation of autophagy-related genes (“ATG”).

As will be appreciated by those skilled in the art inducing autophagy refers to activating the cellular autophagy machinery in a subject such that there is an increase in autophagic flux, for example as evidenced by dynamic biomarkers including an increase in LC3B-II relative to LC3B-I in the presence versus absence of a lysosomal inhibitor, degradation of p62/SQSTM1, and/or enhanced lysosomal activity, which collectively reflect increased formation and lysosomal clearance of autophagosomes. Inducing metabolic conditions associated with autophagy typically refers more broadly to producing systemic and cellular metabolic changes that are known to favor or correlate with autophagy activation, such as reductions in circulating insulin, glucose, and IGF-1, modulation of mTOR and AMPK signaling, and/or elevations in ketone bodies including β-hydroxybutyrate, even where autophagic flux is not directly measured. Thus, in some embodiments the disclosed compositions and regimens induce autophagy as demonstrated by one or more autophagy-specific biomarkers, while in other embodiments the disclosed compositions induce metabolic conditions associated with autophagy as demonstrated by changes in one or more metabolic, hormonal, or ketone markers that are recognized in the art as permissive for, or positively associated with, autophagy. The disclosed regimens can, in at least some formulations, both (i) induce metabolic conditions associated with autophagy (e.g., reduced insulin and IGF-1, elevated ketone bodies, and modulation of mTOR/AMPK signaling) and (ii) induce autophagy itself, as demonstrated by increases in autophagic flux biomarkers such as LC3B-II/LC3B-I in the presence versus absence of a lysosomal inhibitor, p62 degradation, and/or enhanced lysosomal activity in peripheral blood mononuclear cells of subjects consuming the autophagy-inducing dietary compositions.

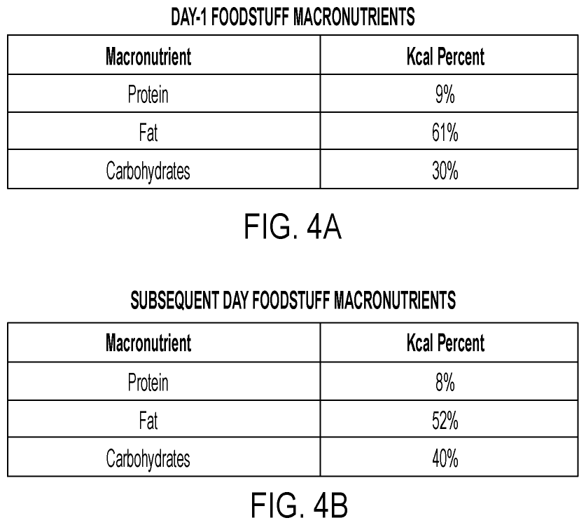

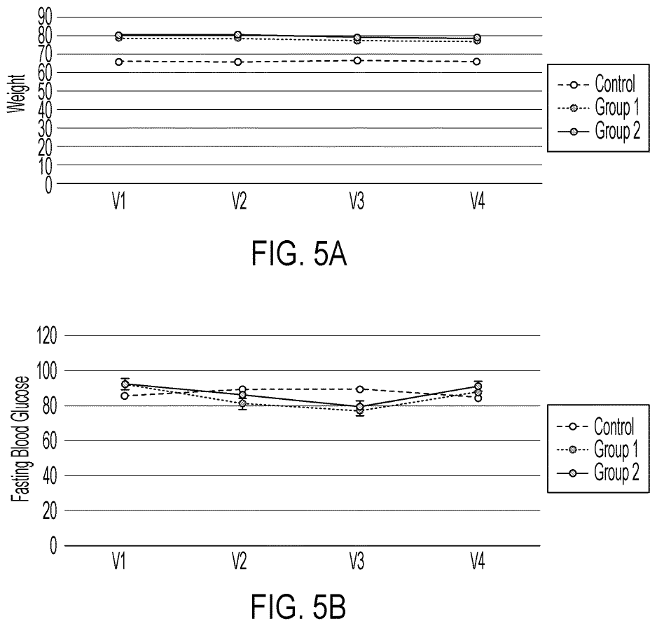

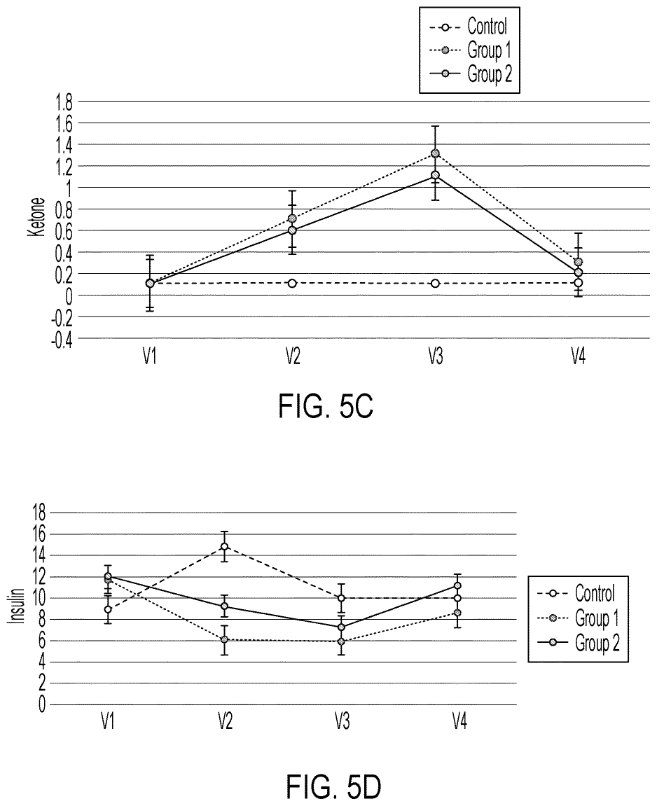

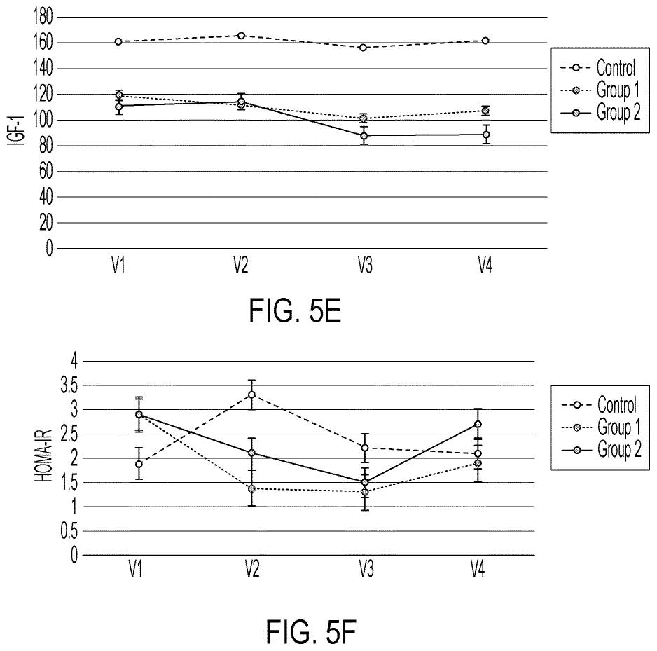

In certain embodiments, a multi-day fasting-mimicking regimen having a first-day dietary formulation and subsequent-day dietary formulation within the macronutrient ranges illustrated in FIGS. 3A-3B and 4A-4B is configured to increase autophagic flux in human peripheral blood mononuclear cells. In particular, in a randomized human clinical study described herein, regimens having a first-day total caloric intake of about 1,000 to 1,150 kcal with about 8% to 11% of kilocalories from protein, about 50% to 57% from fat, and about 34% to 39% from carbohydrate, followed by four subsequent days each providing about 700 to 750 kcal with about 7% to 10% of kilocalories from protein, about 46% to 54% from fat, and about 34% to 39% from carbohydrate, increased LC3B-II to LC3B-I autophagic flux in peripheral blood mononuclear cells by at least 20% at Day 6 relative to baseline and relative to a control diet providing at least about 90% of the subject's normal caloric intake.

Autophagy-inducing dietary compositions and foodstuffs made from the autophagy-inducing dietary compositions are disclosed that support autophagy through a combination of controlled macronutrient intake, specific metabolic signaling, and a structured dietary regimen. Autophagy-inducing dietary compositions can be provided in the form of nutrition bars, soups, powders, beverages, snacks, teas, and/or supplements, ensuring compliance with the dietary regimen while maintaining palatability and convenience. A meal regimen using the foodstuffs manufactured from the autophagy-inducing dietary compositions can span a plurality of days and may, for example, span two, three, four, five, six, seven days or longer.

Autophagy is believed to be induced upon consumption of foodstuffs formulated based on the disclosed autophagy-inducing dietary compositions through multiple interconnected biochemical pathways that sense nutrients, energy, stress, and damage, and that converge on the core ATG machinery for autophagosome formation and lysosomal degradation. Nutrient and energy sensors such as mTORC1 and AMPK are understood in the art to integrate amino acid and ATP/AMP levels to modulate activation of the ULK1-ATG13-FIP200 complex that initiates autophagosome formation. Class III PI3K (Beclin 1-Vps34) complexes may generate phosphatidylinositol 3-phosphate to recruit downstream ATG proteins that drive phagophore nucleation and membrane expansion via ubiquitin-like conjugation systems (ATG12-ATG5-ATG16L1 and LC3/ATG8 lipidation). Stress-activated pathways, including ER stress/UPR, JNK, and Ca²⁺-dependent signaling, are also reported to modulate these core modules, while membrane-trafficking and fusion machinery (SNAREs, tethering factors, and phosphoinositides) completes autophagosome maturation and fusion with lysosomes for cargo degradation, and the metabolic and clinical changes observed with the present regimens are consistent with engagement of one or more of these pathways.

The low-protein content and moderate caloric restriction of the autophagy-inducing dietary compositions suppresses mTOR signaling, which activates ULK1, a key initiator of autophagy. Additionally, the high unsaturated fat content of the autophagy-inducing dietary compositions stimulates AMPK, which further inhibits mTOR and enhances autophagic activity by promoting catabolic energy production. The autophagy-inducing dietary compositions also facilitate ketone production, specifically increasing levels of β-hydroxybutyrate, believed to be an up-regulator of autophagy-related genes. By maintaining a plant-based protein profile, the autophagy-inducing dietary compositions effectively reduce IGF-1 levels, thereby decreasing cellular growth signaling and favoring catabolic pathways such as autophagy. Through these mechanisms, the disclosed autophagy-inducing dietary compositions provide an effective dietary intervention for promoting cellular repair and longevity.

The foodstuffs manufactured from the autophagy-inducing compositions have broad applications for metabolic health, longevity, and disease prevention. Upcoming clinical studies demonstrate that periodic fasting and FMDs can provide significant health benefits for multiple conditions. For individuals with metabolic disorders, the disclosed autophagy-inducing dietary compositions improve insulin sensitivity, glucose homeostasis, and lipid profiles, making it particularly suitable for subjects with prediabetes or metabolic syndrome. In neurodegenerative diseases, the autophagy-inducing dietary compositions and foodstuffs enhance neuronal autophagy, potentially reducing amyloid-beta accumulation in Alzheimer's disease and neuroinflammation in Parkinson's disease. Cardiovascular benefits of consuming foodstuffs manufactured from autophagy-inducing compositions include lowering inflammatory markers, blood pressure, and LDL cholesterol which results in an overall reduction of cardiovascular risk. Furthermore, as a strategy for aging and longevity, the autophagy-inducing compositions support cellular repair and rejuvenation, which delays the onset of age-related diseases. The autophagy-inducing dietary compositions may also serve as an adjuvant in cancer therapy by increasing stress resistance in normal cells while promoting apoptosis in cancerous cells. The present disclosure thus provides a non-pharmaceutical approach to enhancing autophagic flux by consuming foodstuffs manufactured from autophagy-inducing compositions, offering a safe and effective alternative to pharmacological autophagy inducers.

### Multi-Day Meal Regimen

A structured, multi-day meal regimen is disclosed. The structured, multi-day meal regimen mimics intermittent fasting and promotes autophagy in a subject while maintaining essential nutrient intake. The multi-day meal regimen comprises a precisely formulated dietary program that spans e.g., five days, with controlled macronutrient intake. The foodstuffs made from the autophagy-inducing dietary compositions mimic the metabolic effects of fasting without requiring complete food abstinence. However, as will be appreciated by those skilled in the art, the meal regimen itself need not be five days.

For example, on Day 1, the meal regimen provides a controlled caloric intake within a first defined range to initiate metabolic shifts associated with fasting while supplying essential nutrients to the subject. The total caloric intake for Day 1 from foodstuffs manufactured from autophagy-inducing dietary compositions comprise at least, 300 kcal, 400 kcal, 500 kcal, 600 kcal, 700 kcal, 800 kcal, 900 kcal, or 1000 kcal; and at most, 1600 kcal, 1500 kcal, 1400 kcal, 1300 kcal, 1250 kcal, 1200 kcal, 1110 kcal, or 1000 kcal. The total caloric intake for Day 1 may fall within a range of 300 to 1600 kcal, 400 to 1500 kcal, 500 to 1500 kcal, 600 to 1400 kcal, 700 to 1300 kcal, 800 to 1250 kcal, 900 to 1200 kcal, or 1000 to 1150 kcal.

In some examples, the relatively broad total caloric intake ranges for the first-day dietary formulation reflect the recognition that autophagy-permissive metabolic states can be achieved across different absolute calorie levels, provided that protein is restricted and fat and carbohydrate are maintained within the disclosed percentage windows. At lower ends of the Day 1 ranges (e.g., about 300 to 800 kcal), the stronger caloric deficit is expected to rapidly reduce circulating glucose and insulin, lower IGF-1, and initiate ketogenesis, thereby promoting metabolic conditions associated with autophagy. At higher ends of the Day 1 ranges (e.g., about 900 to 1600 kcal), the same macronutrient distributions preserve a low-protein, relatively high-fat pattern that still supports suppression of insulin and IGF-1 and elevation of ketone bodies compared to a subject's normal diet, while improving tolerability and adherence for subjects with higher baseline caloric needs. Across this spectrum, maintaining the macronutrient ratios described herein is expected to produce qualitatively similar fasting-like metabolic responses that favor autophagy, even though the absolute degree of caloric restriction and the magnitude of the biomarker changes may vary between subjects.

The protein content on Day 1 of the meal regimen of the foodstuffs manufactured from autophagy-inducing dietary compositions comprise at least 1%, 2%, 3%, 4%, 5%, 6%, 7%, 8%, or 9% of total caloric intake, and at most 20%, 18%, 16%, 14%, 13%, 12%, 11.5%, or 11% of total caloric intake. The protein content of the meal regimen may be in the range of 1% to 20%, 2% to 18%, 3% to 16%, 4% to 14%, 5% to 13%, 6% to 12%, 7% to 11.5%, or 8% to 11%.

The first-day protein percentage ranges are selected to keep protein intake below levels that strongly activate nutrient-sensing growth pathways, while still providing sufficient essential amino acids for safety and compliance. Protein contributions at or below about 10-12% of total kilocalories are expected to reduce signaling through insulin and IGF-1 and downstream mTOR activation compared to typical Western diets, which often derive 15-20% or more of energy from protein. At the lower end of the disclosed ranges (e.g., about 1-5% of kilocalories), protein is markedly restricted and is therefore expected to further reduce IGF-1 and mTOR signaling. At the upper end of the disclosed ranges (e.g., about 10-20% of kilocalories), the regimens still provide a protein intake that is lower than a normal diet for many subjects, particularly when combined with concurrent caloric restriction, and thus are expected to maintain metabolic conditions that are permissive for or associated with autophagy. These ranges also provide flexibility to tailor protein intake to subject-specific needs (e.g., body weight, baseline diet, or activity level) while preserving the low-protein character of the regimen.

The fat content on Day 1 of the meal regimen of the foodstuffs manufactured from autophagy-inducing dietary compositions comprise at least 30%, 35%, 40%, 42%, 44%, 46%, 48%, 50%, or 52% of total caloric intake, and at most 80%, 75%, 70%, 65%, 63%, 61%, 59%, or 57% of total caloric intake. The fat content may be in the range of 30% to 80%, 35% to 75%, 40% to 70%, 42% to 65%, 44% to 63%, 46% to 61%, 48% to 59%, or 50% to 57%.

The first-day fat percentage ranges are selected to ensure that fat provides a substantial fraction of total kilocalories, thereby supporting a shift toward fatty acid oxidation and ketone body production under conditions of caloric and protein restriction. Fat contributions of at least about 30% of total kilocalories, and in many embodiments 40-60% or more, are expected to promote ketogenesis once hepatic glycogen stores become depleted, increasing circulating ketone bodies such as β-hydroxybutyrate that are associated with autophagy-related signaling and cellular stress resistance. At the higher end of the disclosed ranges (e.g., up to about 70-80% of kilocalories), fat becomes the dominant energy source and can further enhance reliance on lipid metabolism while carbohydrates and protein remain comparatively limited. Across the disclosed ranges, maintaining fat as a primary caloric source under reduced total caloric intake and low protein is expected to contribute to metabolic conditions associated with autophagy while allowing flexibility in formulation and palatability.

The carbohydrate content on Day 1 of the meal regimen of the foodstuffs manufactured from autophagy-inducing dietary compositions comprise at least 10%, 15%, 20%, 25%, 27%, 30%, 32%, 34%, or 36% of total caloric intake, and at most 60%, 55%, 50%, 47%, 45%, 43%, 41%, or 39% of total caloric intake. The carbohydrate content may be in the range of 10% to 60%, 15% to 55%, 20% to 50%, 25% to 47%, 27% to 45%, 30% to 43%, 32% to 41%, or 34% to 39%.

The first-day carbohydrate percentage ranges are designed to limit but not entirely eliminate carbohydrate intake so that subjects can adhere to the regimen while still achieving fasting-like metabolic adaptations. At the lower end of the ranges (e.g., about 10-25% of kilocalories), carbohydrate intake is sufficiently restricted to facilitate depletion of hepatic glycogen and a transition toward increased fatty acid oxidation and ketogenesis, thereby contributing to reductions in glucose and insulin levels. At the higher end of the ranges (e.g., about 40-60% of kilocalories), overall carbohydrate intake remains below that of many baseline diets, particularly when combined with controlled total calories, and may still reduce postprandial glucose and insulin excursions relative to a subject's normal diet while enabling inclusion of fiber-rich, low-glycemic index sources. Thus, within the disclosed carbohydrate percentage windows, and in combination with reduced protein and total calories, the first-day formulation is expected to produce qualitatively similar fasting-like metabolic responses that support metabolic conditions associated with autophagy.

The protein, fat, and carbohydrate content of the meal regimen or any foodstuff manufactured with the disclosed autophagy-inducing dietary compositions can all be controlled independently of each other or together. For example, the meal regimen or foodstuff can have a first day macronutrient profile, when the ranges of macronutrients are all controlled together, of: 300 to 1600 kcal total, with 1% to 20% of kcal from proteins, 30% to 80% from fats, and 10% to 60% from carbohydrates; 400 to 1500 kcal total, with 2% to 18% of kcal from proteins, 35% to 75% from fats, and 15% to 55% from carbohydrates; 500 to 1500 kcal total, with 3% to 16% kcal from proteins, 40% to 70% kcal from fats, and 20% to 50% from carbohydrates; 600 to 1400 kcal total, with 4% to 14% kcal from proteins, 42% to 65% from fats, and 25% to 47% from carbohydrates; 700 to 1300 kcal total, with 5% to 13% kcal from proteins, 44% to 63% from fats, and 27% to 45% from carbohydrates; 800 to 1250 kcal total, 6% to 12% kcal from proteins, 46% to 61% from fats, and 30% to 43% from carbohydrates; 900 to 1200 kcal total, with 7% to 11.5% kcal from proteins, 48% to 59% from fats, and 32% to 41% from carbohydrates; or 1000 to 1150 kcal total, with 8% to 11% kcal from proteins, 50% to 57% from fats, and 34% to 39% from carbohydrates.

The combination of controlled caloric intake and specific macronutrient distribution on Day 1 of the meal regimen is operable to initiate metabolic responses associated with fasting, including shifts in glucose utilization, lipid metabolism, and autophagy activation. The foodstuff components of the meal regimen for Day 1 may include, but are not limited to, nutrition bars, soups, powders, beverages, snacks, teas, and/or supplements, with each foodstuff formulated to align with the defined macronutrient ranges while providing essential micronutrients, fiber, and bioactive compounds that support metabolic adaptation. The macronutrient balance of the foodstuff components consumed by the subject on the day 1 meal regimen allows for the induction of fasting-related benefits for the subject while maintaining sufficient energy intake for adherence and metabolic stability.

On subsequent days, e.g., days 2 to 5, the meal regimen provides a reduced caloric intake to sustain the metabolic shifts initiated for the subject on Day 1 of the meal regimen while further promoting autophagy and cellular adaptation. The total caloric intake for each of the subsequent days comprises at least 200 kcal, 300 kcal, 400 kcal, 450 kcal, 500 kcal, 550 kcal, 600 kcal, 650 kcal, or 700 kcal, and at most 1000 kcal, 950 kcal, 900 kcal, 875 kcal, 850 kcal, 825 kcal, 800 kcal, 775 kcal, or 750 kcal. The total caloric intake for each of Days 2 to 5 may fall within a range of 200 to 1000 kcal, 300 to 950 kcal, 400 to 900 kcal, 450 to 875 kcal, 500 to 850 kcal, 550 to 825 kcal, 600 to 800 kcal, 650 to 775 kcal, or 700 to 750 kcal.

For subsequent days of the regimen, the disclosed total caloric intake ranges provide flexibility to sustain fasting-like metabolism over multiple days in subjects with different baseline energy needs, while maintaining a meaningful caloric deficit relative to normal intake. At lower ends of these ranges (e.g., about 200-500 kcal per day), the regimen approximates a very low-calorie diet that is expected to maintain robust reductions in glucose, insulin, and IGF-1 and to sustain ketone production. At higher ends of the ranges (e.g., about 700-1000 kcal per day), the regimen still typically provides at most about 50% of a subject's normal caloric intake, which is expected to preserve fasting-mimicking metabolic effects while improving tolerability for subjects who are leaner, more active, or otherwise less able to tolerate extreme caloric restriction. Across the disclosed calorie ranges, maintaining the macronutrient profiles described for subsequent days is expected to sustain metabolic conditions associated with autophagy that are initiated on Day 1.

The protein content of autophagy-inducing dietary composition comprises at least 1%, 2%, 3%, 4%, 5%, 6%, 6.5% or 7% of total caloric intake, and at most 15%, 14%, 13%, 12%, 11%, 10.5%, 10.25%, or 10% of total caloric intake. The protein content of the meal regimen foodstuffs may be in the range of 1% to 15%, 2% to 14%, 3% to 13%, 4% to 12%, 5% to 11%, 6% to 10.5%, 6.5% to 10.25%, or 7% to 10%.

The subsequent-day protein percentage ranges continue the low-protein character of the regimen to maintain suppression of growth-promoting signaling pathways over the multi-day period. Protein levels in the range of about 1-10% of kilocalories are expected to further limit activation of the IGF-1 and mTOR axes, particularly when combined with ongoing caloric restriction and high fat intake. The upper bound of about 10-15% of kilocalories accommodates subjects with higher protein requirements, such as older adults or athletes, while still representing a reduction from typical protein contributions in many Western diets and thereby supporting maintenance of metabolic conditions permissive for autophagy. These ranges thus enable clinicians or users to titrate protein intake within a window that preserves the fasting-mimicking and autophagy-supportive characteristics of the regimen.

The fat content of the autophagy-inducing dietary composition comprises at least 30%, 34%, 36%, 38%, 40%, 42%, 44%, or 46% of total caloric intake, and at most 75%, 70%, 65%, 62%, 60%, 58%, 56%, or 54% of total caloric intake. The fat content may be in the range of 30% to 75%, 34% to 70%, 36% to 65%, 38% to 62%, 40% to 60%, 42% to 58%, 44% to 56%, or 46% to 54%.

The subsequent-day fat percentage ranges are chosen to keep fat as the dominant energy source across Days 2-5, thereby supporting continued reliance on fatty acid oxidation and ketogenesis under conditions of reduced total caloric intake. Fat contributions of about 30-75% of kilocalories, and more typically 40-60% or 46-54% of kilocalories in certain preferred embodiments, are expected to maintain elevated ketone bodies such as β-hydroxybutyrate, stabilize energy supply, and support satiety while protein and carbohydrates remain constrained. Sustained high-fat intake in this context, combined with low protein and moderate carbohydrate, is thus expected to conserve lean mass and promote metabolic conditions associated with autophagy over the full course of the regimen.

The carbohydrate content of the autophagy-inducing dietary composition comprises at least 15%, 20%, 23%, 26%, 28%, 30%, 32%, or 34% of total caloric intake, and at most 55%, 52%, 49%, 47%, 45%, 43%, 41%, or 39% of total caloric intake. The carbohydrate content may be in the range of 15% to 55%, 20% to 52%, 23% to 49%, 26% to 47%, 28% to 45%, 30% to 43%, 32% to 41%, or 34% to 39%.

The subsequent-day carbohydrate percentage ranges provide sufficient carbohydrate to support basic physiological needs and permit inclusion of fiber-rich, low-glycemic index foods, while remaining low enough, in view of the reduced total calories and low protein, to maintain decreased glucose and insulin levels relative to a subject's normal diet. At the lower end of the ranges (e.g., about 15-28% of kilocalories), carbohydrate intake is restricted and is expected to favor continued ketogenesis and reliance on fatty acid oxidation. At the higher end (e.g., about 34-55% of kilocalories), carbohydrate intake remains reduced compared to typical diets when total caloric intake is simultaneously lowered, and is expected to avoid strong activation of insulin signaling while improving palatability and adherence. In combination with the specified protein and fat percentages and total caloric intake, these carbohydrate ranges are therefore expected to maintain metabolic conditions associated with autophagy throughout Days 2-5.

The protein, fat, and carbohydrate content of the autophagy-inducing dietary compositions can all be controlled independently of each other or together. For example, the following macronutrient profiles for the foodstuffs for the subsequent days, when the ranges of macronutrients are all controlled together, are: 200 to 1000 kcal total, with 1% to 15% of kcal from proteins, 30% to 75% from fats, and 15% to 55% from carbohydrates; 300 to 950 kcal total, with 2% to 14% of kcal from proteins, 34% to 70% from fats, and 15% to 52% from carbohydrates; 400 to 900 kcal total, with 3% to 13% kcal from proteins, 36% to 65% kcal from fats, and 23% to 49% from carbohydrates; 450to 875 kcal total, 4% to 12% kcal from proteins, 38% to 62% from fats, and 26% to 47% from carbohydrates; 550 to 825 kcal total, with 5% to 11% kcal from proteins, 40% to 60% from fats, and 28% to 45% from carbohydrates; 600 to 800 kcal total, with 6% to 10.5% kcal from proteins, 42% to 58% from fats, and 30% to 43% from carbohydrates; 650 to 775 kcal total, with 6.5% to 10.25% kcal from proteins, 44% to 56% from fats, and 32% to 41% from carbohydrates; or, most preferably, 700 to 750 0 kcal total, 7% to 10% kcal from proteins, 46% to 54% from fats, and 34% to 39% from carbohydrates.

Combinations of foodstuffs which deliver sustained caloric restriction and specific macronutrient distribution when consumed by a subject on, for example, Days 2 to 5 are operable to enhance fasting-associated metabolic responses in the subject, including increased reliance on fatty acid oxidation, ketogenesis, and autophagy activation. The foodstuffs for Days 2 to 5 may include, but are not limited to, nutrition bars, soups, powders, beverages, snacks, teas, and/or supplements, each formulated to align with the defined macronutrient ranges while providing essential micronutrients, fiber, and bioactive compounds that support metabolic adaptation. The macronutrient balance allows for the continuation of fasting-related benefits while providing sufficient energy to support physiological functions.

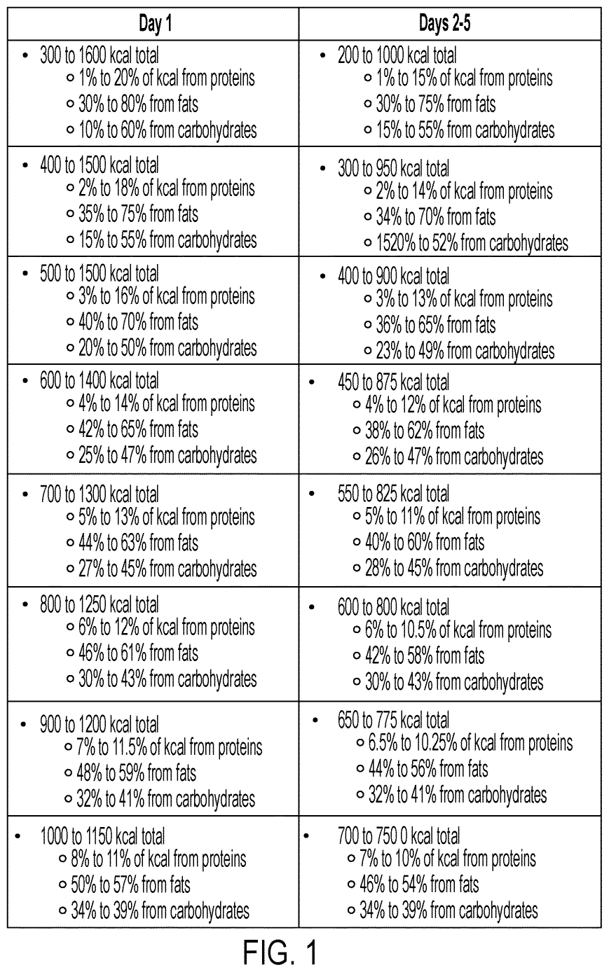

As discussed above, the autophagy-inducing dietary composition macronutrients from the first day and subsequent days may be controlled independently of each other or together. For example, the macronutrient profiles may be proper for the first and subsequent days within a range as shown in FIG. 1.

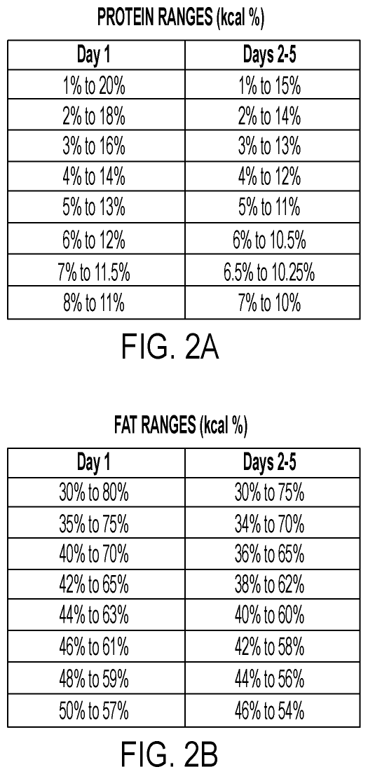

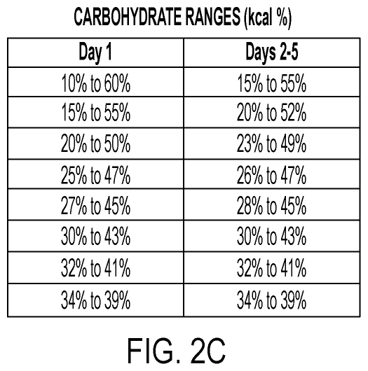

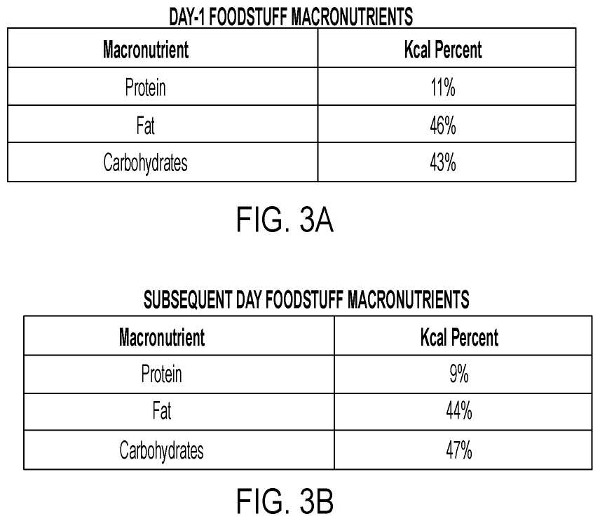

The entire autophagy-inducing dietary composition need not be tied together. For example, individual micronutrients comprising the autophagy-inducing dietary composition may be tied together without other components. FIG. 2A is a table illustrating exemplar protein component ranges by kcal percent for autophagy-inducing dietary compositions. FIG. 2B is a table illustrating exemplar fat components ranges by kcal percent for autophagy-inducing dietary compositions. FIG. 2C is a table illustrating exemplar carbohydrate components ranges by kcal percent for autophagy-inducing dietary compositions.

The meal regimen comprises a variety of nutrition bars, soups, powders, beverages, snacks, teas, and/or supplements, each formulated to align with the macronutrient profile required to sustain the fasting-mimicking state, e.g. a state that mimics the effects of fasting by, for example, providing a subject with about 50% of their normal caloric intake. A meal regimen according to the present disclosure may be formulated to be prepackaged and portioned for ease of use, ensuring compliance of the subject with the dietary protocol. The meal components for each day are selected to maintain low protein intake while providing essential micronutrients, fiber, and bioactive compounds that support cellular stress resistance and repair.

Following a multi-day meal regimen, a refeeding period can be followed by the subject which allows for the gradual reintroduction of a normal caloric intake. The refeeding period is designed to complement the autophagic processes initiated during the multi-day meal regimen during which the foodstuffs formulated according to the disclosed autophagy-inducing dietary compositions are consumed by the subject, supporting metabolic recovery and nutrient replenishment. The meal regimen may be repeated periodically, such as once per month or every other month, to sustain long-term benefits for the subject.

While the multi-day meal regimen is operable to optimize autophagy induction and metabolic benefits while mimicking a fast, the foodstuffs manufactured from the autophagy-inducing dietary compositions may be further adapted for specific physiological and health-related goals of the subject. In certain examples, the autophagy-inducing dietary composition formulation may be modified to accommodate diabetic-specific needs, incorporating lower glycemic index carbohydrates such as fiber-rich legumes, resistant starches, and inulin to promote glucose homeostasis and insulin sensitivity. In another example, the meal regimen may be tailored for neuroprotective applications, including enhanced ketone esters, docosahexaenoic acid, and polyphenol-rich extracts to support cognitive function, neuronal health, and neuroinflammation reduction. Additionally, the formulations for the foodstuffs and the autophagy-inducing dietary compositions in the meal regimen may be adjusted for athlete-focused applications, by, for example, incorporating modifications to protein intake, branched-chain amino acids, or targeted micronutrient supplementation to support muscle retention and recovery while maintaining the metabolic benefits of fasting and autophagy. These adaptations maintain the core principles of the FMD and autophagy while optimizing nutrient profiles for targeted metabolic, neurological, and performance-related benefits of different subjects.

This structured meal regimen provides a practical and effective approach to inducing autophagy while maintaining controlled nutritional support, allowing subjects to experience the cellular benefits of fasting without the challenges of complete food deprivation. The meal regimen provides a foodstuff. The autophagy-inducing dietary compositions of the foodstuffs mimic the metabolic effects of fasting while also supplying essential nutrients that support cellular homeostasis for the subject.

### Autophagy-Inducing Macronutrient Ranges

As described in FIG. 1 and FIGS. 2A-2C, a first-day dietary formulation of a multi-day FMD regimen may provide total caloric intake selected from 300 to 1600 kcal, 400 to 1500 kcal, 500 to 1500 kcal, 600 to 1400 kcal, 700 to 1300 kcal, 800 to 1250 kcal, 900 to 1200 kcal, or 1000 to 1150 kcal. Within each total caloric range, the protein, fat, and carbohydrate components fall within defined kcal percent windows that jointly support fasting-like metabolic responses.

In some configurations, the macronutrient profile for the first-day dietary formulation is:
300 to 1600 kcal total, with 1% to 20% of kcal from proteins, 30% to 80% from fats, and 10% to 60% from carbohydrates;
400 to 1500 kcal total, with 2% to 18% of kcal from proteins, 35% to 75% from fats, and 15% to 55% from carbohydrates;
500 to 1500 kcal total, with 3% to 16% of kcal from proteins, 40% to 70% of kcal from fats, and 20% to 50% from carbohydrates;
600 to 1400 kcal total, with 4% to 14% of kcal from proteins, 42% to 65% from fats, and 25% to 47% from carbohydrates;
700 to 1300 kcal total, with 5% to 13% of kcal from proteins, 44% to 63% from fats, and 27% to 45% from carbohydrates;
800 to 1250 kcal total, with 6% to 12% of kcal from proteins, 46% to 61% from fats, and 30% to 43% from carbohydrates;
900 to 1200 kcal total, with 7% to 11.5% of kcal from proteins, 48% to 59% from fats, and 32% to 41% from carbohydrates; or
1000 to 1150 kcal total, with 8% to 11% of kcal from proteins, 50% to 57% from fats, and 34% to 39% from carbohydrates.

Similarly, the subsequent-day dietary formulation for each of Days 2-5 may provide total caloric intake selected from 200 to 1000 kcal, 300 to 950 kcal, 400 to 900 kcal, 450 to 875 kcal, 500 to 850 kcal, 550 to 825 kcal, 600 to 800 kcal, 650 to 775 kcal, or 700 to 750 kcal, with defined kcal percent windows for protein, fat, and carbohydrates.

The macronutrient profile for the subsequent-day dietary formulation is:
200 to 1000 kcal total, with 1% to 15% of kcal from proteins, 30% to 75% from fats, and 15% to 55% from carbohydrates;
300 to 950 kcal total, with 2% to 14% of kcal from proteins, 34% to 70% from fats, and 15% to 52% from carbohydrates;
400 to 900 kcal total, with 3% to 13% of kcal from proteins, 36% to 65% of kcal from fats, and 23% to 49% from carbohydrates;
450 to 875 kcal total, with 4% to 12% of kcal from proteins, 38% to 62% from fats, and 26% to 47% from carbohydrates;
550 to 825 kcal total, with 5% to 11% of kcal from proteins, 40% to 60% from fats, and 28% to 45% from carbohydrates;
600 to 800 kcal total, with 6% to 10.5% of kcal from proteins, 42% to 58% from fats, and 30% to 43% from carbohydrates;
650 to 775 kcal total, with 6.5% to 10.25% of kcal from proteins, 44% to 56% from fats, and 32% to 41% from carbohydrates; or
700 to 750 kcal total, with 7% to 10% of kcal from proteins, 46% to 54% from fats, and 34% to 39% from carbohydrates.

### Adaptability of Meal Regimen Duration

The meal regimen can be structured to optimize metabolic effects, promote autophagy, and ensure adherence by the subject. The meal regimen is designed to typically span up to five days, which has been identified as a duration that achieves the benefits of fasting while maintaining essential nutrient intake for the subject.

The duration of the meal regimen may be adjusted to accommodate different applications and needs of a subject. The meal regimen can, for example, be formulated for at least: 1 day, 2 days, 3 days, 4 days, or 5 days; and at most 10 days, 11 days, 12 days, 13 days, 14 days, 21 days, 28 days, or 40 days. The duration of the meal regimen may fall within a range of 1 to 40 days, 2 to 28 days, 3 to 21 days, 4 to 14 days, 5 to 13 days, 5 to 12 days, 5 to 11 days, or 5 to 10 days. When the meal regimen is 2 days or longer, the days after day 1 would follow the macronutrient profile as described for the subsequent days (e.g., days 2-5) in the multi-day meal regimen disclosed herein.

The multi-day meal regimen is designed to be repeatable by a subject, with potential for monthly, bimonthly, or quarterly cycles depending on the desired metabolic and health outcomes. A shorter duration of the meal regimen, such as 1 to 4 days, may be implemented for intermittent fasting applications or short-term metabolic resets, whereas an extended meal regimen, such as 6 to 14 days or beyond, may be used by a subject in specialized protocols requiring prolonged autophagy activation.

To maintain metabolic balance in longer meal regimens, a structured refeeding phase may be included to facilitate recovery and nutrient replenishment. The macronutrient distribution and caloric intake of the meal regimen may be modulated based on the duration to sustain fasting-mimicking effects while providing essential dietary support.

A five-day meal regimen is a practical and effective formulation that balances metabolic adaptation, autophagy induction, and nutritional adequacy. This formulation of the dietary components of the meal regimen provides a repeatable, scalable dietary intervention that can be adapted based on individual goals while maintaining principles of a FMD that promotes metabolic adaptation and autophagy induction in a subject.

### Protein Component

The meal regimen incorporates protein sources selected to provide essential amino acids while maintaining a controlled protein intake necessary to support autophagy. The protein content may be derived primarily from plant-based sources, which are selected to minimize activation of nutrient-sensing pathways such as mTOR and IGF-1, thereby promoting the fasting-mimicking state. These plant-based protein sources are balanced to support metabolic function, muscle maintenance, and cellular repair while avoiding excessive protein intake that could interfere with autophagy activation.

Exemplary plant-based protein sources in the formulation may include nuts, seeds, legumes, and vegetables. Nut-derived proteins may be sourced from one or more of, almonds, macadamia nuts, pecans, and/or coconut, which provide both essential amino acids and healthy fats that support metabolic adaptation. Seed-based proteins may be sourced from one or more of, chia seeds, flaxseeds, sesame seeds, and sunflower seeds, offering additional plant-based protein along with fiber and omega-3 fatty acids.

Legume-based proteins can also be included for their favorable amino acid profile and contribution to overall nutrient balance. Representative sources may include white beans, black beans, and peas, which provide a blend of protein and complex carbohydrates while also supporting gut health through their fiber content. Vegetable-based proteins may be derived from kale, spinach, leeks, cabbage, and mushrooms, which contribute essential vitamins, minerals, and phytonutrients in addition to small amounts of protein.

To further enhance nutritional balance, the formulation may include small amounts of fermented protein extracts, yeast-derived proteins, or hydrolyzed plant proteins, which can improve amino acid bioavailability while maintaining the fasting-mimicking effects. These sources help provide an amount of protein suitable to support physiological function without significantly increasing IGF-1 or interfering with autophagy-related metabolic shifts.

The selection of these low-protein, plant-derived sources ensures that the foodstuff delivers essential nutrients while preserving the fasting-induced cellular responses necessary for autophagy, metabolic regulation, and overall health benefits. These protein sources are carefully balanced to sustain energy levels, maintain muscle integrity, and optimize compliance with the fasting-mimicking dietary protocol.

### Fat Component

The meal regimen incorporates fat sources selected to provide sustained energy, support metabolic adaptation, and promote autophagy. The formulation prioritizes healthy fats, particularly unsaturated fats, while limiting protein intake to maintain the fasting-mimicking effects. The inclusion of fats is designed to facilitate ketogenesis, enhance cellular resilience, and regulate nutrient-sensing pathways such as mTOR and AMPK, which play a role in autophagy induction.

Exemplary fat sources in the formulation may include plant-based oils, nuts, seeds, and naturally occurring fats in whole food ingredients. Plant-based oils may include extra virgin olive oil, sunflower oil, and coconut oil, which provide monounsaturated and polyunsaturated fats essential for metabolic flexibility and sustained energy. Olive oil, in particular, is a rich source of oleic acid, which has been associated with beneficial effects on inflammation and cellular repair processes. Olive oil may be in the form of olive oil, virgin olive oil, or extra virgin olive oil.

Nut-derived fats may include macadamia nuts, almonds, pecans, and coconut, which offer a blend of monounsaturated and polyunsaturated fats, as well as small amounts of naturally occurring saturated fats that contribute to energy balance. These sources also contain fat-soluble vitamins and bioactive compounds that support cellular homeostasis.

Seed-based fats may be derived from flaxseeds, chia seeds, and sesame seeds, which provide omega-3 and omega-6 fatty acids that contribute to membrane integrity, inflammatory modulation, and metabolic function. These sources also supply lignans and other phytochemicals that may support antioxidant defense mechanisms.

Additional sources of dietary fat may include cocoa butter and naturally occurring fats in vegetables such as olives and avocados, which contribute to a balanced lipid profile that supports ketosis and fasting metabolism. The formulation may also include medium-chain triglycerides (“MCTs”) derived from coconut oil, which are rapidly converted into ketone bodies, further enhancing metabolic flexibility and the fasting-mimicking state.

By prioritizing unsaturated and plant-derived fats, the foodstuff provides a sustained source of energy while maintaining autophagy-promoting effects. The careful selection of fat sources ensures that the meal regimen supports metabolic efficiency, enhances adherence, and contributes to the overall health benefits associated with fasting-mimicking diets.

### Carbohydrate Component

The meal regimen includes carefully selected carbohydrate sources to provide sustained energy, support metabolic flexibility, and enhance autophagy while maintaining a low glycemic impact or low glycemic index. The carbohydrates in the foodstuff are primarily derived from fiber-rich, complex, and low-glycemic index sources that minimize insulin spikes and optimize glucose metabolism. These carbohydrates contribute to overall nutrient balance and facilitate adherence to the meal regimen by enhancing satiety and digestive health.

Exemplary carbohydrates in the formulation may include whole grains, legumes, vegetables, and fiber sources. Representative whole grain sources include quinoa, whole grain brown rice, and oats, which provide essential micronutrients and sustained energy release. Legume-derived carbohydrates may include white beans, peas, and black beans, providing a combination of complex carbohydrates and plant-based protein while supporting gut health through their prebiotic fiber content.

Vegetable-derived carbohydrates provide additional fiber, antioxidants, and phytonutrients, contributing to cellular protection and metabolic adaptation. Representative vegetable sources may include carrots, spinach, kale, leeks, zucchini, champignon mushrooms, and cabbage. Additionally, the formulation may include root and tuber-derived carbohydrates such as potato flakes, which provide resistant starch that supports gut microbiota and metabolic flexibility.

Additional carbohydrate sources may include natural sweeteners and plant-derived fibers. Natural sweeteners, such as honey, coconut sugar, and grape juice, may be included in controlled amounts to enhance palatability without significantly impacting glycemic load. Fiber sources, such as inulin, chicory root fiber, tapioca flour, and rice dextrin, contribute to digestive health, promote satiety, and further modulate the metabolic response to fasting.

The selection of these carbohydrates ensures that the foodstuff provides necessary metabolic support while maintaining the desired autophagic response and fasting-mimicking effects. These sources collectively contribute to a balanced nutrient profile that sustains energy levels, enhances compliance, and optimizes cellular health.

### Micronutrients and Additives

In addition to fats, proteins, and carbohydrates, the meal regimen and/or foodstuff may include a variety of additives as micronutrients, bioactive compounds, and functional additives that enhance metabolic adaptation, support cellular processes, and promote adherence to the meal regimen. These components may be selected to optimize nutrient absorption, maintain electrolyte balance, and provide antioxidant, anti-inflammatory, or gut-supporting properties.

Dietary fiber plays a critical role in digestive health, glycemic control, and metabolic stability during fasting-mimicking periods. Fiber may be included to enhance satiety, promote gut microbiota diversity, and support intestinal health. Representative fiber sources include inulin, chicory root fiber, flaxseed meal, resistant starch, and plant-derived insoluble fibers. The inclusion of fiber helps modulate blood sugar levels, supports bowel regularity, and contributes to the overall metabolic benefits of the meal regimen.

Maintaining electrolyte balance is essential during fasting-mimicking periods to prevent dehydration, muscle cramps, and metabolic imbalances. The composition may include sodium (e.g., sea salt), potassium (e.g., potassium citrate or potassium chloride), magnesium (e.g., magnesium chloride or magnesium sulfate), and calcium (e.g., calcium citrate or calcium carbonate) to support cellular hydration, neuromuscular function, and metabolic efficiency.

Additional trace minerals such as zinc, selenium, iron, iodine, copper, manganese, chromium, and molybdenum may be included to promote enzyme function, antioxidant defense, and immune system support.

Gut microbiota composition can influence immune function, metabolic health, and nutrient absorption. The fasting-mimicking composition may include probiotic strains (e.g., Lactobacillus, Bifidobacterium) and prebiotic fibers (e.g., inulin, oligofructose) to promote a balanced gut microbiome and support intestinal barrier integrity during the fasting-mimicking period.

To provide comprehensive nutritional support while maintaining fasting-mimicking effects, the formulations, compositions and/or foodstuffs may include water-soluble and fat-soluble vitamins. Examples of vitamins that may be included are: Vitamin A (as mixed carotenoids or retinol); Vitamin C (ascorbic acid or sodium ascorbate); Vitamin D (cholecalciferol); Vitamin E (as mixed tocopherols); Vitamin K (as K1 or K2); and B-complex vitamins (B1 thiamine, B2 riboflavin, B3 niacin, B5 pantothenic acid, B6 pyridoxine, B7 biotin, B9 folate, B12 methylcobalamin or cyanocobalamin). These vitamins may be incorporated to support metabolic pathways, mitochondrial function, cellular repair, and immune regulation.

To enhance cellular resilience and counteract oxidative stress, the formulations, compositions, and/or foodstuffs may also include natural antioxidants and bioactive compounds. Representative compounds include: polyphenols (from sources such as rosemary extract, green tea extract, cocoa, or turmeric); flavonoids (from sources such as citrus peel, hibiscus, and red peppers); carotenoids (from sources such as tomatoes, sweet potatoes, and carrots); and sulfur-containing compounds (from sources such as onions, garlic, and cruciferous vegetables). These compounds may help modulate inflammatory responses, enhance cellular detoxification, and provide neuroprotective and cardioprotective benefits.

To improve palatability and adherence, the formulations, compositions, and/or foodstuffs may contain natural flavors, herbal extracts, and mild sweeteners, especially those that do not significantly impact blood glucose levels. Representative examples include: herbs and spices (e.g., basil, oregano, rosemary, thyme, cumin, chili pepper, turmeric, lemongrass); cocoa derivatives (e.g., cocoa powder, cocoa mass, cocoa butter); citrus extracts (e.g., lemon peel, orange peel); and mild natural sweeteners (e.g., monk fruit extract, stevia, erythritol, or controlled amounts of honey or coconut sugar). Additionally, citric acid, ascorbic acid, and mixed tocopherols may be included as natural preservatives to maintain the freshness and stability of the food components.

To support ketogenesis and metabolic flexibility, the formulations, compositions, and/or foodstuffs may optionally include MCTs from coconut oil or palm kernel oil. MCTs are rapidly converted into ketone bodies, which provide an alternative energy source and enhance the fasting-mimicking metabolic state. Additionally, compounds such as beta-hydroxybutyrate salts or ketone esters may be incorporated to further promote ketone production and metabolic efficiency.

The inclusion of fiber, electrolytes, vitamins, antioxidants, probiotics, flavor enhancers, and ketone-supporting compounds in the formulations, compositions, and/or foodstuffs may ensure that the meal regimen provides a nutritionally complete yet metabolically restrictive composition. These additional components contribute to sustained energy levels, metabolic balance, and cellular resilience while preserving the fasting-mimicking state necessary for auto31phagy induction and long-term metabolic benefits.

### Exemplary Formulations

In one group (Group 1, Formulation A, corresponds to the commercially available ProLon® regimen which provides approximately 11% of kcal from protein, 46% from fat, and 43% from carbohydrates on Day 1 and approximately 9% protein, 44% fat, and 47% carbohydrates on Days 2-5), the FMD regimen provides on Day 1 about 1100 kcal with about 11% of kcal from protein, about 46% of kcal from fat, and about 43% of kcal from carbohydrates. On each of Days 2-5, the regimen provides about 720 kcal with about 9% of kcal from protein, about 44% of kcal from fat, and about 47% of kcal from carbohydrates. [ProLon® is a registered trademark of L-Nutra Inc. for dietary bars and foodstuffs.]

In another group (Group 2, a second FMD formulation, previously evaluated under the development name ‘ProMete,’ is referred to herein as Formulation B and is characterized by approximately 9% protein kcal, 61% fat kcal, and 30% carbohydrate kcal on Day 1 and approximately 8% protein kcal, 52% fat kcal, and 40% carbohydrate kcal on Days 2-5.), the FMD regimen provides on Day 1 about 9% of kcal from protein, about 61% of kcal from fat, and about 30% of kcal from carbohydrates, and on each of Days 2-5 about 8% of kcal from protein, about 52% of kcal from fat, and about 40% of kcal from carbohydrates.

The autophagy-inducing dietary compositions can be formulated into foodstuffs such as nutrition bars, soups, snacks, powders, beverages, teas, and supplements, including combinations thereof, and provided as pre-portioned components in daily meal boxes labeled for Days 1-5 of the regimen.

### Mechanistic Basis

The disclosed autophagy-inducing compositions and FMD regimens are designed to trigger autophagy through multiple interconnected biochemical pathways involving nutrient-sensing and energy homeostasis. Low overall caloric intake and low protein content suppress mTOR signaling and promote activation of ULK1, while high unsaturated fat content and resultant increases in β-hydroxybutyrate activate AMPK and upregulate autophagy-related genes.

The macronutrient profiles, plant-based protein sources, and cyclic fasting-refeeding pattern are configured to reduce IGF-1 levels, favor catabolic over anabolic pathways, enhance ketogenesis, and promote cellular repair processes associated with autophagy.

### Manufacturing

The foodstuff may be manufactured using low-temperature processing techniques designed to preserve the bioactivity of key ingredients while ensuring food safety, stability, and compliance with the intended nutritional formulation. Each step of the manufacturing process can be optimized to prevent nutrient degradation, maintain product consistency, and protect sensitive bioactive compounds that contribute to the metabolic and autophagy-inducing effects of the formulation. The low-temperature processing may be conducted at a temperature not exceeding about 45 °C, about 42 °C, about 40 °C, about 38 °C, about 36 °C, about 34 °C, or about 32 °C. In certain embodiments, the low-temperature processing is conducted at a temperature of at least about −20 °C, about −18 °C, about −15 °C, about −12 °C, about −10 °C, about −8 °C, or about −5 °C. In certain embodiments, the processing temperature ranges from about −20 °C to about 45 °C, from about −18 °C to about 42 °C, from about −15 °C to about 40 °C, from about −12 °C to about 38 °C, from about −10 °C to about 36 °C, from about −8 °C to about 34 °C, or from about −5 °C to about 32 °C

The production process may begin with sourcing and selecting high-quality, plant-based ingredients that align with the proprietary macronutrient formulation. Ingredients can be inspected for purity, quality, and absence of contaminants, helping to ensure that the final product meets regulatory and nutritional standards. After selection, ingredients may be blended in specific proportions to achieve the desired macronutrient balance, maintaining appropriate levels of protein, fats, and carbohydrates to support the fasting-mimicking effect.

To preserve nutrient integrity, different food components can be processed using tailored techniques. Nutrition bars and snacks may be manufactured using a cold-press extrusion technique, which can minimize exposure to heat and reduce the risk of degrading temperature-sensitive nutrients such as polyphenols, omega fatty acids, and vitamin complexes.

For powdered components, including dehydrated soups, vegetable-based blends, and plant-based protein formulations, low-temperature dehydration, freeze-drying, or spray-drying methods may be used. In some cases, drum drying or vacuum drying may also be applied, depending on the properties of the ingredients. The dehydration process can begin by carefully washing and chopping fresh ingredients, which may then be steam-blanched to reduce microbial load and deactivate enzymes that could degrade flavor and nutritional quality. The prepared ingredients can then be subjected to one or more dehydration techniques. Freeze-drying, or lyophilization, involves freezing the prepared food ingredients and then gradually reducing atmospheric pressure to sublimate ice directly into vapor. The freeze-drying, or lyophilization process can preserve heat-sensitive compounds, maintain structural integrity, and result in powders that rapidly rehydrate when mixed with liquid. Spray-drying may be used for liquid-based ingredients such as broth or plant-based emulsions, where a fine mist of the liquid formulation is sprayed into a heated chamber, causing rapid evaporation of water and leaving behind a powdered product. The spray-drying technique can be used for flavor components, vitamins, and lipid-encapsulated ingredients. Drum drying can be used for vegetable or legume-based formulations, where the prepared puree is spread onto a heated drum, dried into a thin sheet, and then ground into a powder. The drum drying approach can help produce smooth, quick-rehydrating powders for soups and porridge-like formulations. Vacuum drying may be used for heat-sensitive ingredients, such as probiotic blends or bioactive compounds, which are dried under reduced pressure at lower temperatures to minimize thermal degradation while preserving functional properties.

After dehydration, the resulting dried ingredients can be milled or ground into a fine, uniform powder. To ensure proper flowability and prevent caking, anti-caking agents such as silica or maltodextrin may be incorporated in small amounts. For some formulations, microencapsulation techniques may be used to coat lipophilic ingredients, vitamins, or probiotics with protective layers to enhance their stability and bioavailability. Once powdered components are prepared, they may be blended with additional seasonings, fiber sources, or micronutrient fortifications to achieve the desired nutritional and sensory profile. The final powder formulations can then be packaged in moisture-resistant, oxygen-barrier pouches to protect against humidity and oxidation. Vacuum-sealing or nitrogen flushing may be employed to further enhance shelf stability.

Through this controlled dehydration and powder-processing approach, fasting-mimicking food components, including soups and other meal formulations, can be produced in a format that is lightweight, shelf-stable, and convenient for reconstitution, while maintaining the intended nutritional and metabolic benefits.

### Kits, Packaging and Methods of Use

A kit comprises: (a) a plurality of pre-portioned foodstuffs manufactured from autophagy-inducing dietary compositions having a first-day dietary formulation and at least one subsequent-day dietary formulation as described herein; and (b) packaging and labeling instructions specifying sequential daily consumption over a multi-day regimen.

The foodstuffs formed from the autophagy-inducing dietary composition may be provided as part of a prepackaged multi-day meal kit (fasting-mimicking dietary kit), wherein the foodstuffs for each day are individually portioned and labeled for sequential consumption. To maintain the integrity of the composition and enhance compliance, the dietary kit includes individual packaging for the foodstuff corresponding to the formulation for the day, ensuring proper macronutrient distribution and adherence to the meal regimen.

To protect against oxidation, moisture intrusion, and nutrient degradation, the packaging may be engineered with multiple protective features. Vacuum-sealed pouches may be used to minimize oxygen exposure, reducing oxidative degradation of sensitive fats and bioactive compounds. In some examples, nitrogen-flushed packaging replaces oxygen with an inert gas to extend shelf life and prevent rancidity in high-unsaturated-fat components. Desiccant or moisture-absorbing inserts may be included to control humidity and prevent degradation of dried components, such as soups, powders, and nutrition bars. Additionally, multi-layer barrier films, such as foil-laminated polymer films, may be utilized to block oxygen and light, maintaining the stability of sensitive micronutrients and phytochemicals. Resealable containers are unnecessary, as the regimen is strictly controlled, with each day's intake carefully pre-portioned for compliance with the fasting-mimicking protocol.

The dietary kit may also include supplemental formulations, such as electrolyte blends, ketone precursors, or functional beverages, packaged separately to complement the fasting-mimicking state. Additionally, color-coded or sequentially numbered packaging may be employed to ensure users consume the formulations in the intended order. In some examples, specific formulations may be provided for particular dietary needs, such as diabetic-friendly, neuroprotective, or athlete-focused variations, ensuring precise macronutrient composition and functional ingredient inclusion.

Through these packaging innovations, the dietary kit provides enhanced stability of the packaged foodstuffs, prolonged shelf life of the packaged foodstuffs, ensuring optimal nutritional efficacy, and ease of use for the subject which increases compliance with the multi-day regimen.

Methods of inducing autophagy in a subject comprise administering the kit or FMD regimen to the subject monthly, bimonthly, or at another periodic interval, thereby providing repeated cycles of autophagy induction and metabolic benefits over time. Methods of treating or reducing risk of an age-related disease, metabolic disorder, cardiovascular condition, neurodegenerative disease, inflammatory disorder, autoimmune disease, or cancer in a subject comprise administering one or more cycles of the disclosed FMD regimen to the subject, optionally in combination with standard-of-care therapies are also enabled.

### EXAMPLE 1

In a first example, the autophagy-inducing composition is formulated to be consumed as part of a structured multi-day dietary regimen. In this example, the meal regimen includes 1100 kcal on Day 1 from the foodstuff manufactured from the autophagy-inducing components having the percentages shown in FIG. 3A.

In this example, the meal regimen provides approximately 720 kcal per day from Days 2 to 5 from the foodstuff manufactured from the autophagy-inducing components having the percentages shown in FIG. 3B. After completing a five-day cycle, the subject resumes their normal diet during the refeeding period.

Ingredients used to create the meal regimen include: almonds, annatto extract (color), ascorbic acid, basil, black pepper, broccoli, brown rice crispy, brown rice syrup, cabbage, carrot, celery, champignon mushroom, chia seeds, chili pepper, chocolate, citric acid, cocoa, cumin seed, flaxseed, garlic, gelatin, glycerin, grape juice, honey, inulin, kale, lactic acid, leeks, lemon peel, lemongrass, macadamias, natural flavor, oats, olive oil, olives, onion, oregano, parsley, peas, pecans, potassium sorbate (to protect freshness), potato, quinoa, rice dextrin, rice flour, rosemary extract, sea salt, sesame seeds, soy lecithin, spinach, sunflower oil, sweet red peppers, tapioca flour, thyme, tomato, turmeric, white beans, whole grain brown rice, yeast extract, zucchini squash.

Supplementals may include: algal oil, vitamin A (as mixed carotenoids), vitamin C (as ascorbic acid), vitamin D (as cholocalciferol), vitamin E (as mixed tocopherols), vitamin K1 (as phytonadione), vitamin B1 (as thiamin hydrochloride), vitamin B2 (as riboflavin), vitamin B3 (as niacinamide), vitamin B5 (as calcium pantothenate), vitamin B6 (as pyridoxal-5-phosphate), folic acid (as calcium folinate), vitamin B12 (as methylcobalamin), biotin, calcium (as calcium citrate), iron (as ferrous bis-glycinate), iodine (as potassium iodide), magnesium (as magnesium chloride), zinc (as zinc citrate), selenium (as l-selenomethionine), copper (as copper gluconate), manganese (as manganese sulfate), chromium (as chromium picolinate), molybdenum (as sodium molybdate). other ingredients: hypromellose, microcrystalline cellulose, magnesium stearate, silicon dioxide.

This example has been evaluated for its ability to induce metabolic conditions associated with autophagy in a human subject through biochemical and metabolic markers. To confirm autophagy induction, the ratio of LC3B-II to LC3B-I protein in peripheral blood mononuclear cells was measured using Western blot analysis before and after treatment with an autophagy-blocking agent, chloroquine (“CQ”). A significant increase in the CQ-treated to untreated LC3B-II/LC3B-I ratio was observed following consumption of the meal regimen, particularly on Day 6, indicating an enhancement in autophagic flux.

Additionally, the degradation of protein p62, a well-established marker of autophagic activity, was monitored, with a notable decrease confirming increased autophagic degradation. Metabolic markers further supported autophagy activation, as fasting plasma β-hydroxybutyrate levels were significantly elevated while insulin and glucose levels were reduced, indicative of a metabolic shift toward ketogenesis and nutrient-sensing pathway modulation. Lysosomal activity assays also demonstrated increased enzymatic function, corroborating enhanced autophagic processing.

These findings confirmed that the structured dietary regimen described herein effectively induce metabolic conditions associated with autophagy in human subjects, providing a non-pharmaceutical intervention for cellular renewal, metabolic health improvement, and longevity support. The observed metabolic shifts align with expected fasting physiology, further validating the structured approach of the present disclosure. However, these findings do not limit the disclosure to any specific mechanistic pathway.

This periodic fasting regime may be repeated on a monthly or bimonthly basis to maintain metabolic benefits and promote long-term autophagic activation. Through this structured dietary approach, the present disclosure provides a practical, science-backed solution for promoting autophagy, improving metabolic health, and extending health span.

### EXAMPLE 2

In a second example, the autophagy-inducing composition is designed to be consumed as part of a structured multi-day dietary regimen. Here, the meal regimen includes 1100 kcal on Day 1 from the foodstuff manufactured from the autophagy-inducing components having the percentages shown FIG. 4A.

According to this example, the meal regimen provides approximately 720 kcal per day from Days 2 to 5 from the foodstuff manufactured from the autophagy-inducing components having the percentages shown in FIG. 4B. After completing a five-day cycle, the subject resumes their normal diet during the refeeding period.

Ingredients used to create the meal regimen include: almond, basil, black beans, black pepper, carrot, celery, champignon mushrooms, chia seeds, chicory root fiber, chili pepper, cilantro, citric acid, coconut flour, coconut sugar, coconut vinegar, cumin seed, extra virgin olive oil, flaxseed, garlic, hibiscus, honey, kale, leek, lemon peel, lemongrass, macadamia nuts, mixed tocopherols (vitamin E), natural flavor, olives, onion, oregano, parsley, peas, pecans, potato flakes, rice flour, rice starch, rosemary, rosemary extract, savoy cabbage, sea salt, sesame seeds, spearmint, spinach, sunflower oil, sweet red peppers, tapioca flour, tomato, turmeric, white beans, yeast extract.

Supplementals may include: algal oil, vitamin A (as mixed carotenoids), vitamin C (as ascorbic acid), vitamin D (as cholocalciferol), vitamin E (as mixed tocopherols), vitamin K1 (as phytonadione), vitamin B1 (as thiamin hydrochloride), vitamin B2 (as riboflavin), vitamin B3 (as niacinamide), vitamin B5 (as calcium pantothenate), vitamin B6 (as pyridoxal-5-phosphate), folic acid (as calcium folinate), vitamin B12 (as methylcobalamin), biotin, calcium (as calcium citrate), iron (as ferrous bis-glycinate), iodine (as potassium iodide), magnesium (as magnesium chloride), zinc (as zinc citrate), selenium (as l-selenomethionine), copper (as copper gluconate), manganese (as manganese sulfate), chromium (as chromium picolinate), molybdenum (as sodium molybdate). other ingredients: hypromellose, microcrystalline cellulose, magnesium stearate, silicon dioxide.

This example has also been evaluated for its ability to induce autophagy through biochemical and metabolic markers. To confirm autophagy induction, the ratio of LC3B-II to LC3B-I protein in peripheral blood mononuclear cells was measured using Western blot analysis before and after treatment with an autophagy-blocking agent, chloroquine (“CQ”). A significant increase in the CQ-treated to untreated LC3B-II/LC3B-I ratio was observed following consumption of the meal regimen, particularly on Day 6, indicating an enhancement in autophagic flux.

Additionally, the degradation of p62, a well-established marker of autophagic activity, was monitored, with a notable decrease confirming increased autophagic degradation. Metabolic markers further supported autophagy activation, as fasting plasma β-hydroxybutyrate levels were significantly elevated while insulin and glucose levels were reduced, indicative of a metabolic shift toward ketogenesis and nutrient-sensing pathway modulation. Lysosomal activity assays also demonstrated increased enzymatic function, corroborating enhanced autophagic processing.

These findings confirm that the structured dietary regimen described herein effectively induce metabolic conditions associated with autophagy, providing a non-pharmaceutical intervention for cellular renewal, metabolic health improvement, and longevity support. The observed metabolic shifts align with expected fasting physiology, further validating the structured approach of the present disclosure. However, these findings do not limit the disclosure to any specific mechanistic pathway.

This periodic fasting regimen may be repeated on a monthly or bimonthly basis to maintain metabolic benefits and promote long-term autophagic activation. Through this structured dietary approach, the present disclosure provides a practical, science-backed solution for promoting autophagy, improving metabolic health, and extending health span.

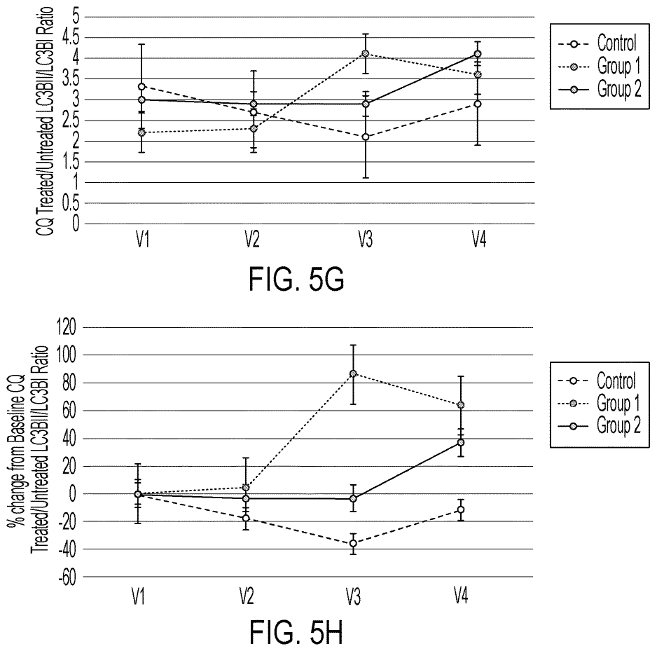

### Clinical Results (FIGS. 5A-5H)

### Human Clinical Study

A randomized, open-label, three-arm clinical trial was conducted in thirty healthy adult participants to evaluate the effects of two FMD formulations (Group 1 (Formulation A), Group 2 (Formulation B)) compared to a control diet on autophagic flux and metabolic health markers over an 8-day study period. The thirty participants were recruited from the community. At an initial screening visit, medical history, physical exam, electrocardiogram, and clinical labs (complete blood count (CBC), comprehensive metabolic panel (CMP), and lipid panel), were assessed to determine eligibility. Exclusion criteria based on medical history, recent weight loss interventions, weight loss medication, therapies, pregnancy, allergies and alcohol dependency were evaluated prior to inclusion in the study.

Participants were 49.1+11.8 years (range: 25-65 years) and 83% female. In the first group there was a significant increase in the CQ treated: untreated LCBH/LC3B-I ratio compared to baseline, although there was not a significant difference when considering all groups. Both FMD groups showed a significant decrease in insulin (p=0.002), glucose (p=0.0003), and HOMA-IR (p=0.001), and an increase in ketone BHB (p=0.0005) compared to control.

Participants were randomized 1:1:1 to control, Group 1 FMD, or Group 2 FMD, and consumed their assigned regimen for five days, with blood draws at baseline (Visit 1), Day 4 (Visit 2), Day 6 (Visit 3), and 48 hours post-intervention at Day 8 (Visit 4). Autophagic flux in peripheral blood mononuclear cells was quantified as the ratio of LC3B-II/LC3B-I protein in chloroquine (CQ)-treated samples to untreated samples.

Metabolic outcomes included fasting plasma glucose, insulin, insulin-like growth factor (IGF) IGF-1, β-hydroxybutyrate (BHB), and HOMA-IR, with statistical analyses including ANOVA or Kruskal-Wallis tests for group differences, paired tests for within-subject changes, and multivariate linear mixed effects models for longitudinal effects.

Turning to FIG. 5A, the weight of the control group, Group 1 and Group 2 remained substantially the same from Visit 1 (V1) to Visit 4 (V4). The Fasting Blood Glucose (FIG. 5B) remained relatively stable for the control group over V1 to V4, while the Fasting Blood Glucose if Group 1 was lower than the Fasting Blood Glucose of Group 2 over the same period. Ketone measurement, FIG. 5C, was stable for the control group, while it increased sharply in both Group 1 and Group 2 to V3, before decreasing at V4. In FIG. 5D, the insulin levels for the control group increase from V1 to V2, before reducing at V3 and remaining steady to V4. In contrast the insulin levels for Group 1 and Group 2 have a starting point that is higher than the control group, but follow a downward trend (Group 2) before increasing at V4, or a downward trend (Group 1) which is stable before increasing at V4. In FIG. 5E, the IGF-1 level for the control group is fairly level, while the IGF-1 levels in Group 1 and Group 2 are below the control group with Group 2 achieving a lower level than Group 1.

Turning to FIG. 5F, the HOMA-IR levels for the control group increase from V1 to V2, before reducing at V3 and V4. In contrast the HOMA-IR levels for Group 1 and Group 2 have a starting point that is higher than the control group, but follow a downward trend (Group 2) before increasing at V4, or a downward trend (Group 1) which is stable before increasing at V4. In FIG. 5G, the CQ Treated/Untreated LC3B-II/LC3B-I Ratio for the control group decreases to its lowest level at V3, while the ration increased to a top rate at V3 for Group 1, and a top rate at V4 for Group 2. In FIG. 5H, the Percent change from Baseline CQ Treated/Untreated LC3B-II/LC3B-I Ratio for Group 1 and Group 2 have similar progressions at the CQ Treated/Untreated ratios shown in FIG. 5G.

FIGS. 5A-5H summarize clinical test data from a randomized, controlled trial evaluating two multi-day fasting-mimicking regimens based on the disclosed autophagy-inducing dietary compositions in human subjects. In this study, subjects were randomized to a control arm that continued a normal diet providing at least about 90% of the subject's estimated normal caloric intake, or to a fasting-mimicking diet arm that consumed a five-day regimen within the macronutrient ranges illustrated in FIGS. 3A-3B and 4A-4B. In one example, the fasting-mimicking regimen (Formulation A) provided on Day 1 a total of about 1,000 to 1,150 kcal with about 8% to 11% of kilocalories from protein, about 50% to 57% from fat, and about 34% to 39% from carbohydrate, and on each of Days 2-5 a total of about 700 to 750 kcal with about 7% to 10% of kilocalories from protein, about 46% to 54% from fat, and about 34% to 39% from carbohydrate.

Autophagic flux in peripheral blood mononuclear cells (PBMCs) was assessed at baseline, Day 4, Day 6, and Day 8 (48 hours post-intervention) by measuring LC3B-II and LC3B-I protein levels via Western blot in the presence and absence of chloroquine (CQ) ex vivo. Autophagic flux was quantified as the ratio of CQ-treated to untreated LC3B-II/LC3B-I, a validated measure of autophagic degradation activity.

In the absence of CQ, a higher LC3B-II/LC3B-I ratio reflects increased autophagic induction, whereas a lower ratio suggests elevated autophagic degradation. While subjects in the control arm exhibited an increase of 0.5±0.7 units in CQ-untreated LC3B-II/LC3B-I ratio at Day 6, the ones receiving the fasting-mimicking diet formulation (Formulation A, ProLon) showed a decrease in the LC3B-II/LC3B-I ratio from 1.1±0.6 at baseline to 0.7±0.4 at Day 6 (mean change −0.3±0.4). This decrease indicates enhanced autophagic degradation. The between-arm difference in change from baseline to Day 6 was statistically significant across both fasting-mimicking diet formulations relative to control (p=0.0035, Kruskal-Wallis test).

The ratio of LC3B-II/LC3B-I in CQ-treated versus CQ-untreated samples is a better measure of autophagic flux, the combined rate of autophagic induction and degradation. The ProLon group showed an increase of autophagic flux at Day 6 (mean change 1.9±2.9 vs. −1.1±2.0 in the control group). This elevation persists through Day 8 following refeeding, supporting sustained enhancement of autophagic flux beyond the active dietary intervention period. For the FMD2 group, the elevated trend in autophagy flux was observed 2 days post dietary intervention.

The observed increase in autophagic degradation and flux is consistent with fasting-mimicking diet-induced modulation of nutrient-sensing pathways, including inhibition of mTOR signaling and activation of AMPK-dependent processes, resulting in increased lysosomal turnover of intracellular components.

These data demonstrate that a fasting-mimicking dietary intervention can enhance autophagic flux in human subjects in the absence of pharmacological agents, supporting its use as a non-drug modality for regulating cellular recycling pathways associated with metabolic health and aging.

FIG. 5A illustrates changes in LC3B-II/LC3B-I autophagic flux ratio between baseline and Day 6 for the control arm and the Formulation A fasting-mimicking arm. FIGS. 5B-5H illustrate corresponding changes in metabolic and hormonal markers, including β-hydroxybutyrate, fasting glucose, fasting insulin, IGF-1, HOMA-IR, body weight, and lipid parameters. In the fasting-mimicking arm, the five-day regimen produced concurrent increases in β-hydroxybutyrate and decreases in fasting glucose, insulin, and IGF-1 relative to baseline and control, consistent with induction of metabolic conditions associated with autophagy in addition to the observed increase in autophagic flux.

Across all intervention groups, significant differences in autophagic markers were observed between baseline and Day 4 (Visit 2). At Day 6 (Visit 3), untreated LC3B-II/LC3B-I levels differed significantly among groups, with higher values in the control group indicative of reduced autophagic protein usage compared to FMD groups.

In mixed effects models, Group 1 (Formulation A) exhibited a statistically significant increase in chloroquine-treated to untreated LC3B-II/LC3B-I ratio at Day 6 (Visit 3) relative to control, with an estimated increase of about 3.1±1.1 units. These data demonstrate that at least one FMD formulation within the disclosed macronutrient ranges induces increased autophagic flux (LC3B-II/LC3B-I+CQ) in human PBMCs over a multi-day regimen having the disclosed first-day and subsequent-day macronutrient windows.

Collectively, the data reflected in FIGS. 5A-5H support that (i) the disclosed FMD regimens significantly improve markers of metabolic health and (ii) at least one disclosed formulation increases autophagic flux in human subjects.

As will be appreciated by those skilled in the art a group or class of materials for a given purpose in connection with the disclosure includes mixtures of any two or more of the members of the group or class as equally suitable. Additionally, description of constituents in chemical terms includes constituents at the time of addition to any combination disclosed herein, and does not necessarily preclude chemical interactions among the constituents of a mixture once mixed.

It will also be appreciated by those skilled in the art that integer ranges include intervening integers. For example, the integer range 1-10 includes 1, 2, 3, 4, 5, 6, 7, 8, 9, and 10. Similarly, the range 1 to 100 includes 1, 2, 3, 4 . . . 97, 98, 99, 100. Similarly, when a range is described, intervening numbers that are increments of the difference between the upper limit and the lower limit divided by 10 can be taken as alternative upper or lower limits. For example, if the range is 1.1 . . . to 2.1 the following numbers 1.2, 1.3, 1.4, 1.5, 1.6, 1.7, 1.8, 1.9, and 2.0 can be selected as lower or upper limits. In the specific examples set forth herein, formulations can be practiced with plus or minus 50 percent of the values indicated rounded to three significant figures. In a refinement, formulations can be practiced with plus or minus 30 percent of the values indicated rounded to three significant figures of the value provided in the examples. In another refinement, formulations can be practiced with plus or minus 10 percent of the values indicated rounded to three significant figures of the value provided in the examples.

As will be appreciated by those skilled in the art, throughout this specification, unless the context requires otherwise, the word “comprise” or variations such as “comprises” or “comprising”, will be understood to imply the inclusion of a stated integer or group of integers but not the exclusion of any other integer or group of integers.

The present teachings may also extend to one or more of the following numbered clauses:

Clause 1. A dietary regimen comprising: a first-day autophagy-inducing dietary composition with a first-day dietary formulation containing 300 to 1600 kcal and comprising protein in an amount of 1 to 20% by kcal; fat in the amount of 30 to 80% by kcal; and carbohydrate in the amount of 10 to 60% by kcal; and a subsequent-day autophagy-inducing dietary composition with a subsequent-day dietary formulation containing 200 to 1000 kcal and comprising proteins in the amount of 1 to 15% by kcal; fats in the amount of 30 to 75% by kcal; and carbohydrates in the amount of 15 to 55% by kcal.

Clause 2. The dietary regimen of clause 1, wherein there are at least 2 subsequent days.

Clause 3. The dietary regimen of clause 1 or 2, wherein there are 4 subsequent days.

Clause 4. The dietary regimen of clause 1 to 3, wherein the first-day autophagy-inducing dietary composition and the subsequent-day autophagy-inducing dietary composition are operable to promote metabolic conditions associated with autophagy in a subject when consumed over a multi-day regimen.

Clause 5. The dietary regimen of clause 1 to 4, wherein the first-day dietary formulation contains 300 to 1600 kcal and comprises proteins in the amount of 5 to 13% by kcal, fats in the amount of 44 to 63% by kcal, and carbohydrates in the amount of 27 to 45% by kcal; and wherein the subsequent-day dietary formulation for at least one subsequent day contains 550 to 825 kcal and each subsequent-day dietary formulation comprises proteins in the amount of 5 to 11% by kcal, fats in the amount of 40 to 60% by kcal, and carbohydrates in the amount of 28 to 45% by kcal.

Clause 6. The dietary regimen of clause 1 to 5, wherein there are at least 2 subsequent days.

Clause 7. The dietary regimen of clause 1 to 6, wherein there are 4 subsequent days.

Clause 8. The dietary regimen of clause 7, wherein the first-day autophagy-inducing dietary composition and the subsequent-day autophagy-inducing dietary composition are operable to promote a metabolic condition associated with autophagy in a subject when consumed over a multi-day regimen.

Clause 9. The dietary regimen of clause 1 to 8, wherein the first-day dietary formulation comprises protein in an amount of 7 to 11.5 of a kcal percent of an entire first-day dietary formulation, and wherein the subsequent-day dietary formulations each contain protein in an amount of 6.5 to 10.25 of a kcal percent of the subsequent-day dietary formulation.

Clause 10. The dietary regimen of clause 1 to 9, wherein the first-day dietary formulation comprises fat in an amount of 48 to 59 of a kcal percent of the entire first-day dietary formulation, and wherein the subsequent-day dietary formulations each contain fat in an amount of 44 to 56 of a kcal percent of the subsequent-day dietary formulation.

Clause 11. The dietary regimen of clause 1 to 5, wherein the first-day dietary formulation comprises carbohydrate in an amount of 32 to 41 of a kcal percent of the entire first-day dietary formulation, and wherein the subsequent-day dietary formulations each contain carbohydrate in an amount of 32 to 41 of a kcal percent the subsequent-day dietary formulation.

Clause 12. A dietary kit, comprising: a first-day autophagy-inducing dietary composition with a first-day dietary formulation containing 300 to 1600 kcal and comprising proteins in an amount of 1 to 20% by kcal; fats in the amount of 30 to 80% by kcal; and carbohydrates in the amount of 10 to 60% by kcal; and a subsequent-day autophagy-inducing dietary composition with a subsequent-day dietary formulation containing 200 to 1000 kcal and comprising proteins in the amount of 1 to 15% by kcal; fats in the amount of 30 to 75% by kcal; and carbohydrates in the amount of 15 to 55% by kcal; and packaging operable to separately contain the first-day autophagy-inducing dietary composition and each subsequent-day autophagy-inducing dietary composition, wherein the first-day autophagy-inducing dietary composition and the subsequent-day autophagy-inducing dietary composition are pre-portioned into individual daily packages corresponding to a specific day of a multi-day regimen, and further wherein each package is labeled for sequential consumption over the multi-day regimen.

Clause 13. The dietary kit of clause 12, wherein the packaging contains sub-packaging that separately contains the first-day autophagy-inducing dietary composition and each subsequent-day autophagy-inducing dietary composition and is labeled.

Clause 14. The dietary kit of clause 12 or 13, wherein the first-day dietary formulation contains 300 to 1600 kcal and comprises proteins in the amount of 5 to 13% by kcal, fats in the amount of 44 to 63% by kcal, and carbohydrates in the amount of 27 to 45% by kcal, and further wherein the subsequent-day dietary formulation for at least one subsequent day contains 550 to 825 kcal and each subsequent-day dietary formulation comprises proteins in the amount of 5 to 11% by kcal, fats in the amount of 40 to 60% by kcal, and carbohydrates in the amount of 28 to 45% by kcal.

Clause 15. A method of inducing autophagy in a subject, the method comprising: administering to the subject a autophagy-inducing dietary composition comprising a first-day autophagy-inducing dietary composition with a first-day dietary formulation containing 300 to 1600 kcal and comprising proteins in an amount of 1 to 20% by kcal; fats in the amount of 30 to 80% by kcal; and carbohydrates in the amount of 10 to 60% by kcal, and a subsequent-day autophagy-inducing dietary composition with a subsequent-day dietary formulation containing 200 to 1000 kcal and comprising proteins in the amount of 1 to 15% by kcal; fats in the amount of 30 to 75% by kcal; and carbohydrates in the amount of 15 to 55% by kcal of clause 1 to 10 over a multi-day regimen, wherein the subject consumes the first-day autophagy-inducing dietary composition on a first day and at least one subsequent-day dietary formulation on at least one subsequent day, thereby inducing metabolic conditions associated with autophagy.

Clause 16. The method of clause 15, wherein the subject consumes the subsequent-day autophagy-inducing dietary composition for at least 2 days after consuming the first-day autophagy-inducing dietary composition.

Clause 17. The method of clause 15, wherein the first-day dietary formulation contains 300 to 1600 kcal and comprises proteins in the amount of 5 to 13% by kcal, fats in the amount of 44 to 63% by kcal, and carbohydrates in the amount of 27 to 45% by kcal, and wherein the subsequent-day dietary formulation for at least one subsequent day contains 550 to 825 kcal and each subsequent-day dietary formulation comprises proteins in the amount of 5 to 11% by kcal, fats in the amount of 40 to 60% by kcal, and carbohydrates in the amount of 28 to 45% by kcal.

Clause 18. A method of manufacturing a fasting-mimicking autophagy-inducing dietary composition, the method comprising: selecting and combining ingredients into at least one first foodstuff to achieve a first macronutrient formulation corresponding to a first-day dietary formulation containing 300 to 1600 kcal and comprising proteins in an amount of 1 to 20% by kcal; fats in the amount of 30 to 80% by kcal; and carbohydrates in the amount of 10 to 60% by kcal, and at least one second foodstuff to achieve a second macronutrient formulation corresponding to a subsequent-day autophagy-inducing dietary composition with a subsequent-day dietary formulation containing 200 to 1000 kcal and comprising proteins in the amount of 1 to 15% by kcal; fats in the amount of 30 to 75% by kcal; and carbohydrates in the amount of 15 to 55% by kcal; packaging the at least one first foodstuff and the at least one second foodstuff into individual daily portions for at least 2 daily portions; and labeling the packaging.

Clause 19. The method of clause 18, wherein the at least one first foodstuff and the at least one second foodstuff comprise one or more of: a nutrition bar, a soup, a powder, a beverage, a snack, a tea, or a supplement.

Clause 20. The method of clause 18 or 19, wherein each of the at least one first foodstuff and the at least one second foodstuff is apportioned into five individual portions.

Clause 21. The method of clause 18 to 20, wherein the first-day dietary formulation contains 300 to 1600 kcal and comprises proteins in the amount of 5 to 13% by kcal, fats in the amount of 44 to 63% by kcal, and carbohydrates in the amount of 27 to 45% by kcal, and wherein the subsequent-day dietary formulation for at least one subsequent day contains 550 to 825 kcal and each subsequent-day dietary formulation comprises proteins in the amount of 5 to 11% by kcal, fats in the amount of 40 to 60% by kcal, and carbohydrates in the amount of 28 to 45% by kcal.

Clause 22. A method of inducing autophagic flux in a human subject, the method comprising: administering to the subject, on a first day of a multi-day fasting-mimicking regimen, one or more foodstuffs providing a total of 1,000 to 1,150 kilocalories, wherein 8% to 11% of total kilocalories are from protein, 50% to 57% are from fat, and 34% to 39% are from carbohydrate; and administering to the subject, on each of four subsequent consecutive days, one or more foodstuffs providing a total of 700 to 750 kilocalories, wherein 7% to 10% of total kilocalories are from protein, 46% to 54% are from fat, and 34% to 39% are from carbohydrate; wherein the fasting-mimicking regimen increases autophagic flux in peripheral blood mononuclear cells of the subject, as evidenced by an increase of at least 20% in a chloroquine-treated to untreated LC3B-II/LC3B-I protein ratio measured after completion of the fifth day relative to baseline and to a control diet providing at least 90% of the normal caloric intake of the subject.

Although these teachings have been described with respect to various examples, it should be realized these teachings are also capable of a wide variety of further and other examples within the spirit and scope of these teachings. Thus, although an overview of the subject matter has been described with reference to specific examples, various modifications and changes may be made to these examples without departing from the broader scope of the present disclosure. It will also be obvious to those skilled in the art that the examples are provided by way of example only. Numerous variations, changes, and substitutions will occur to those skilled in the art without departing from the scope of the disclosure. It should also be understood that various alternatives may be employed in practicing the disclosed innovation. It is intended that any claims presented at any time in this application, or a subsequent application, define the scope of the invention and that methods and structures within the scope of any claims and their equivalents are covered thereby.

What is claimed is:

1. A multi-day dietary regimen for inducing metabolic conditions associated with autophagy in a human subject, the regimen comprising:
administering to the human subject on a first day a first-day dietary formulation providing from 300 to 1600 kcal, the first-day dietary formulation comprising proteins in an amount of 1% to 20% of total kcal, fats in an amount of 30% to 80% of total kcal, and carbohydrates in an amount of 10% to 60% of total kcal; and
administering to the human subject on at least one subsequent day a subsequent-day dietary formulation providing from 200 to 1000 kcal, the subsequent-day dietary formulation comprising proteins in an amount of 1% to 15% of total kcal, fats in an amount of 30% to 75% of total kcal, and carbohydrates in an amount of 15% to 55% of total kcal;
wherein the regimen is configured to induce metabolic conditions associated with autophagy in the human subject.

2. The multi-day dietary regimen of claim 1, wherein the first-day dietary formulation provides from 1000 to 1150 kcal, with proteins in an amount of 8% to 11% of total kcal, fats in an amount of 50% to 57% of total kcal, and carbohydrates in an amount of 34% to 39% of total kcal.

3. The multi-day dietary regimen of claim 1, wherein each subsequent-day dietary formulation provides from 700 to 750 kcal, with proteins in an amount of 7% to 10% of total kcal, fats in an amount of 46% to 54% of total kcal, and carbohydrates in an amount of 34% to 39% of total kcal.

4. The multi-day dietary regimen of claim 1, wherein the regimen spans 5 consecutive days comprising the first day and four subsequent days.

5. The multi-day dietary regimen of claim 1, wherein foodstuffs providing the first-day and subsequent-day dietary formulations are plant-based and low in protein, overall calories, and sugars, and high in unsaturated fats.

6. The multi-day dietary regimen of claim 1, wherein the regimen is repeated periodically at monthly or bimonthly intervals.

7. The multi-day dietary regimen of claim 1, wherein administration of the regimen to the human subject increases autophagic flux in peripheral blood mononuclear cells, as measured by an increase in a chloroquine-treated to untreated LC3B-II/LC3B-I protein ratio relative to baseline.

8. The multi-day dietary regimen of claim 7, wherein the regimen comprises administering a first FMD formulation having about 11% protein kcal, about 46% fat kcal, and about 43% carbohydrate kcal on Day 1, and about 9% protein kcal, about 44% fat kcal, and about 47% carbohydrate kcal on each of Days 2-5.

9. The multi-day dietary regimen of claim 1, wherein administration of the regimen to the human subject reduces at least one metabolic health marker selected from fasting plasma glucose, fasting insulin, IGF-1, and HOMA-IR, and increases β-hydroxybutyrate, compared to a control diet.

10. The multi-day dietary regimen of claim 1, wherein the regimen reduces biological age, immune system age, or both in the human subject.

11. A kit for inducing metabolic conditions associated with autophagy in a human subject, the kit comprising:
a first set of pre-portioned foodstuffs manufactured from autophagy-inducing dietary compositions configured to provide a first-day dietary formulation according to claim 1 or 2; and
a second set of pre-portioned foodstuffs manufactured from autophagy-inducing dietary compositions configured to provide at least one subsequent-day dietary formulation according to claim 1;
wherein the kit further comprises packaging and labeling instructing sequential daily consumption of the first set on a first day and the second set on one or more subsequent days.

12. The kit of claim 11, wherein the foodstuffs comprise one or more of nutrition bars, soups, powders, beverages, snacks, teas, and supplements.

13. The kit of claim 11, wherein the kit is configured for 5 consecutive days of consumption.

14. The kit of claim 11, wherein the kit is configured for periodic use at monthly or bimonthly intervals.

15. A method of improving a metabolic health marker in a human subject in need thereof, the method comprising administering to the human subject the multi-day dietary regimen of claim 1, thereby improving at least one metabolic health marker selected from fasting plasma glucose, fasting insulin, IGF-1, β-hydroxybutyrate, and HOMA-IR.

16. The method of claim 15, wherein the human subject is overweight, obese, or has metabolic syndrome, prediabetes, or type 2 diabetes.

17. A method of inducing autophagy in a human subject, the method comprising administering to the human subject the multi-day dietary regimen of claim 1, thereby increasing autophagic flux in the human subject.

18. The method of claim 17, wherein autophagic flux is assessed by measuring a chloroquine-treated to untreated LC3B-II/LC3B-I ratio in peripheral blood mononuclear cells before and after administration of the regimen.

19. A method of treating or reducing risk of an age-related disease, neurodegenerative disease, cardiovascular disease, inflammatory disease, autoimmune disease, or cancer in a human subject, the method comprising administering to the human subject one or more cycles of the multi-day dietary regimen of claim 1.

20. A method of manufacturing a fasting-mimicking autophagy-inducing dietary composition, the method comprising:
selecting and combining ingredients into at least one first foodstuff to achieve a first macronutrient formulation corresponding to a first-day dietary formulation according to claim 1; and
selecting and combining ingredients into at least one second foodstuff to achieve a second macronutrient formulation corresponding to a subsequent-day dietary formulation according to claim 1;
packaging the at least one first foodstuff and the at least one second foodstuff into individual daily portions for at least two daily portions; and
labeling the packaging to indicate a sequence of consumption.

21. A method of inducing autophagic flux in a human subject, the method comprising:
administering to the human subject, on a first day of a multi-day fasting-mimicking regimen, one or more foodstuffs providing a total of 1,000 to 1,150 kilocalories, wherein 8% to 11% of total kilocalories are from protein, 50% to 57% are from fat, and 34% to 39% are from carbohydrate; and
administering to the human subject, on each of four subsequent consecutive days, one or more foodstuffs providing a total of 700 to 750 kilocalories, wherein 7% to 10% of total kilocalories are from protein, 46% to 54% are from fat, and 34% to 39% are from carbohydrate;
wherein the fasting-mimicking regimen increases autophagic flux in peripheral blood mononuclear cells of the human subject, as evidenced by an increase of at least 20% in a chloroquine-treated to untreated LC3B-II/LC3B-I protein ratio measured after completion of the fifth day relative to baseline and to a control diet providing at least 90% of the normal caloric intake of the human subject.
